(19) United States
(12) Patent Application Publication
(10) Pub. No.: US 2024/0055099 A1
(43) Pub. Date: Feb. 15, 2024

(54) RANGE OF MOTION DETERMINATION

(71) Applicant: Bardavon Health Digital, Inc., Overland Park, KS (US)

(72) Inventors: Jeffrey Miles Greenberg, San Francisco, CA (US); Renhao Wang, Toronto (CA); Manish Shah, San Francisco, CA (US); Borja Arias Drake, Rijeka (HR); Emmett Jackson Greenberg, Mill Valley, CA (US); Oriol Janes Pereira, Barcelona (ES)

(21) Appl. No.: 18/496,438

(22) Filed: Oct. 27, 2023

Related U.S. Application Data
(63) Continuation-in-part of application No. 17/717,074, filed on Apr. 9, 2022.
(60) Provisional application No. 63/173,340, filed on Apr. 9, 2021.

(51) Int. Cl. G16H 20/30; G16H 30/20; A61B 5/11; A61B 5/00

(52) U.S. Cl. CPC G16H 20/30; G16H 30/20; A61B 5/1128; A61B 5/4833; A61B 5/742

(57) ABSTRACT

A method for computer-assisted exercise is described. The method includes loading a definition for the exercise including an ordered list of steps. Each step includes positions, and each position is defined by constraints. For each step in the ordered list, the method loads the current step, receives an image of a patient performing the exercise; and determines a plurality of keypoints in the image. For each constraint, a determination is made as to whether the constraint is being met by the patient based, at least in part, on the relative locations of the plurality of keypoints. In response to determining a first set of constraints in the position is met by the patient, the method proceeds to a next step in the ordered list of steps. Methods for authoring the definitions for the exercises are also described.

### STATEMENT OF RELATED INVENTIONS

This application is a Continuation-in-Part of U.S. U.S. Nonprovisional Application Ser. No. 17/717,074, filed Apr. 9, 2022, which in turn claims the benefit of U.S. Provisional Application No. 63/173,340, filed Apr. 9, 2021, and both are hereby incorporated by reference in their entirety.

### SUMMARY

The below summary is merely representative and non-limiting.

In a first aspect, an embodiment provides a method for computer-assisted exercise. The method includes loading a definition for the exercise including an ordered list of steps. Each step includes at least one position, and each position is defined by at least one constraint. For each step in the ordered list of steps, the method performs loading a current step, receiving an image of a patient performing the exercise; and determining a plurality of keypoints in the image. For each constraint of the at least one constraint of each position, a determination is made as to whether the constraint is being met by the patient based, at least in part, on the relative locations of the plurality of keypoints. In response to determining a first set of constraints in the position is met by the patient, the method proceeds to a next step in the ordered list of steps.

In another aspect, an embodiment provides a method for computer-assisted definition of an exercise. The method includes receiving a description of at least one position. The position defines a step and an ordered list of steps defines the exercise. For each position, the method includes determining a plurality of keypoints in an armature for the position, assigning at least one constraint to the position based, at least in part, on the relative locations of the plurality of keypoints and assigning a first set of constraints which, when met, indicate the associated position has been performed. The method also includes storing the at least one constraint as a position in a step; and storing at least one step as part of the exercise. The method includes the organization of the constraints into aggregates available for search and for reorganization into other aggregates that define an exercise and aggregates of exercises.

### BRIEF DESCRIPTION OF THE DRAWINGS

Aspects of the described embodiments are more evident in the following description, when read in conjunction with the attached Figures.

FIG. 1 is a diagram illustrating an environment in which some embodiments may operate.

FIG. 2 is a diagram illustrating an environment in which some embodiments may operate.

FIG. 3 is a diagram illustrating a method of guiding a person through conformant exercise that may be performed in some embodiments.

FIGS. 4A and 4B, collectively referred to as FIG. 4, show a diagram illustrating a method of obtaining an exercise care plan for a patient from a library and of a way for patient to be guided to conform to the exercise conformance specification that may be performed in some embodiments.

FIG. 5 is a diagram illustrating a jitter smoothing method that may be performed in some embodiments.

FIG. 6 is a diagram illustrating key points corresponding to the joints of the body which some embodiments may use.

FIG. 7 is a diagram illustrating key points connected to form an armature.

FIG. 8 is a diagram illustrating an armature in different positions.

FIG. 9 is a diagram illustrating comparing armatures.

FIGS. 10A and 10B are each a diagram illustrating accelerometer sensor data encoded as an image.

FIG. 11 is a diagram illustrating recognizing movement in order to improve the accuracy of the model.

FIG. 12 is a diagram illustrating an environment in which some embodiments may operate.

FIG. 13 shows a process flow for a coach application analysis.

FIG. 14 shows a process flow for a step matching method.

FIG. 15 illustrates a step of uploading an exercise video.

FIG. 16 illustrates a step of defining a position.

FIG. 17 illustrates selecting a position from a list of sample positions.

FIG. 18 illustrates entering definitions for a position.

FIG. 19 illustrates entering a warning for a position.

FIG. 20 illustrates an example of a routine creation.

FIG. 21 illustrates entering details for an exercise.

FIG. 22 illustrates entering speech/text which may be used during an exercise.

FIG. 23 shows a process flow for an armature recognition.

FIG. 24 demonstrates a sample armature recognition.

FIG. 25 shows a system architecture having the Movement Conformance Engine.

FIG. 26 shows the Movement Conformance Engine in closer detail.

FIG. 27 illustrates using a natural language prompt to generate an exercise plan.

### DETAILED DESCRIPTION

In this specification, reference is made in detail to specific embodiments of the Movement Conformance Engine. Some of the embodiments or their aspects are illustrated in the drawings.

For clarity in explanation, the Movement Conformance Engine has been described with reference to specific embodiments, however it should be understood that the Movement Conformance Engine is not limited to the described embodiments. On the contrary, the Movement Conformance Engine covers alternatives, modifications, and equivalents as may be included within its scope as defined by any patent claims. The following embodiments of the Movement Conformance Engine are set forth without any loss of generality to, and without imposing limitations on, the claimed Movement Conformance Engine. In the following description, specific details are set forth in order to provide a thorough understanding of the present Movement Conformance Engine. The present Movement Conformance Engine may be practiced without some or all of these specific details. In addition, well known features may not have been described in detail to avoid unnecessarily obscuring the Movement Conformance Engine.

In addition, it should be understood that steps of the methods set forth can be performed in different orders than the order presented in this specification. Furthermore, some steps of the methods may be performed in parallel rather than being performed sequentially. Also, the steps of the methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

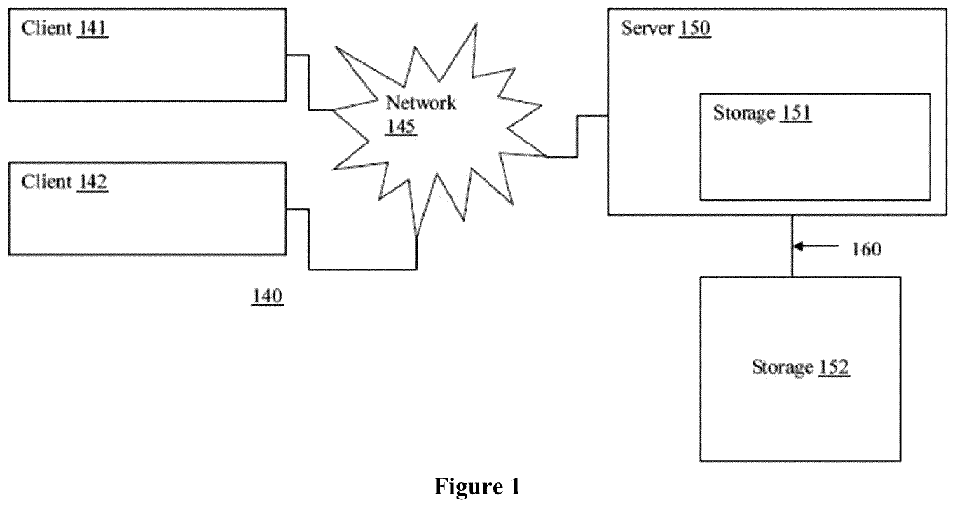

A diagram of a network environment in which embodiments may operate is shown in FIG. 1. In the environment 140, two clients 141, 142 are connected over a network 145 to a server 150 having local storage 151. Clients and servers in this environment may be computers. Server 150 may be configured to handle requests from clients.

The environment 140 is illustrated with only two clients and one server for simplicity, though in practice there may be more or fewer clients and servers. The computers have been termed clients and servers, though clients can also play the role of servers and servers can also play the role of clients. In some embodiments, the clients 141, 142 may communicate with each other as well as the servers. Also, the server 150 may communicate with other servers.

The network 145 may be, for example, local area network (LAN), wide area network (WAN), telephone networks, wireless networks, intranets, the Internet, or combinations of networks. The server 150 may be connected to storage 152 over a connection medium 160, which may be a bus, crossbar, network, or other interconnect. Storage 152 may be implemented as a network of multiple storage devices, though it is illustrated as a single entity. Storage 152 may be a file system, disk, database, or other storage.

In an embodiment, the client 141 may perform the method 200 or other method herein and, as a result, store a file in the storage 152. This may be accomplished via communication over the network 145 between the client 141 and server 150. For example, the client may communicate a request to the server 150 to store a file with a specified name in the storage 152. The server 150 may respond to the request and store the file with the specified name in the storage 152. The file to be saved may exist on the client 141 or may already exist in the server's local storage 151. In another embodiment, the server 150 may respond to requests and store the file with a specified name in the storage 151. The file to be saved may exist on the client 141 or may exist in other storage accessible via the network such as storage 152, or even in storage on the client 142 (e.g., in a peer-to-peer system).

In accordance with the above discussion, embodiments can be used to store a file on local storage such as a disk or on a removable medium like a flash drive, CD-R, or DVD-R. Furthermore, embodiments may be used to store a file on an external storage device connected to a computer over a connection medium such as a bus, crossbar, network, or other interconnect. In addition, embodiments can be used to store a file on a remote server or on a storage device accessible to the remote server.

Furthermore, cloud computing is another example where files are often stored on remote servers or remote storage systems. Cloud computing refers to pooled network resources that can be quickly provisioned so as to allow for easy scalability. Cloud computing can be used to provide software-as-a-service, platform-as-a-service, infrastructure-as-a-service, and similar features. In a cloud computing environment, a user may store a file in the “cloud,” which means that the file is stored on a remote network resource though the actual hardware storing the file may be opaque to the user.

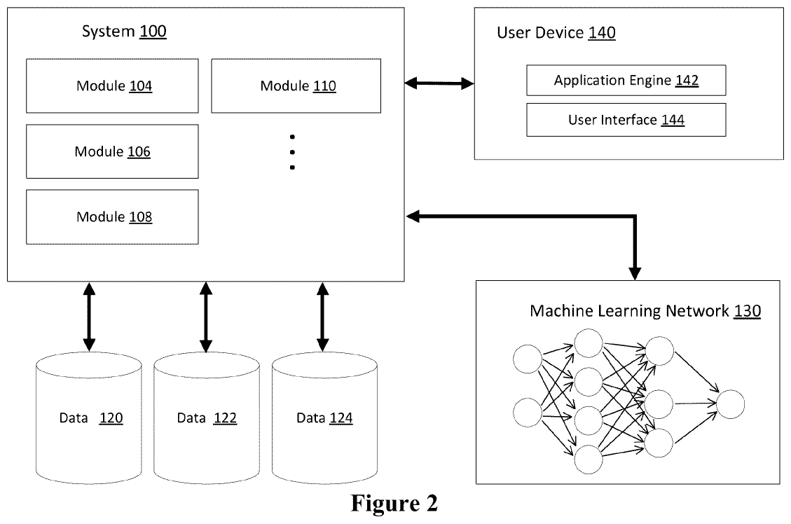

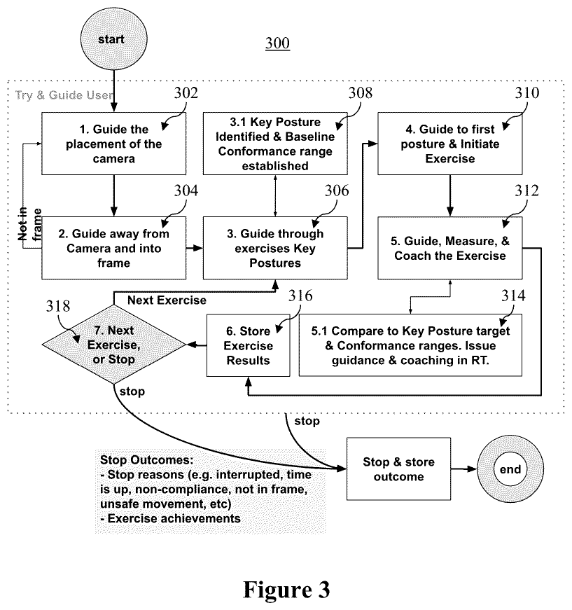

FIG. 2 illustrates a block diagram of an example system 100 for a Movement Conformance Engine that includes respective modules 104, 106, 108, 110 . . . for initiating, implementing and executing any of the operations, steps, methods, processing, data capture, data generation and/or data presentation described herein and illustrated by any of FIGS. 3, 4, 5, 6, 7, 8, 9, 10A, 10B and/or 11. The system 100 may communicate with a user device 140 to display output, via a user interface 144 generated by an application Movement Conformance Engine 142.

While the databases 120, 122 and 124 are displayed separately, the databases and information maintained in a database may be combined together or further separated in a manner the promotes retrieval and storage efficiency and/or data security. It is understood that, in various embodiments, one or more of the modules 104, 106, 108, 110 . . . may reside and be implemented on the user device 140. In addition, respective portions of one or more of the modules 104, 106, 108, 110 . . . may reside and be implemented on the user device 140, while other portions of the same one or more modules 104, 106, 108, 110 . . . may reside and be implemented remotely from the user device 140.

As shown flowchart 300 of FIG. 3, the Movement Conformance Engine instructs the user to place a computing device in a location such that a camera(s) associated with the computing device can capture video/images of the user performing various types of exercises. (Act 302) In various embodiments, the Movement Conformance Engine may include voice and/or user interface prompts providing instructions to the user to place the computing device at a certain location or when to place the device at a certain location. It is understood that the embodiments described herein may allow for the user to hold the computing device during performance of an exercise(s) as opposed to placing it at a particular location.

In some embodiments, the Movement Conformance Engine captures accelerometer and/or gyroscope data to determine whether the computing device is positioned in a proper orientation (e.g., vertical orientation, horizontal orientation). In various embodiments, the Movement Conformance Engine executes one or more machine learning models and/or augmented reality (AR) processing to detect and/or recognize characteristics of the physical environment of the user to determine whether the user has enough open space available to perform one or more exercises.

The Movement Conformance Engine further provides prompts and/or instructions to guide the user away from the placed computing device and to select a placement for themselves in relation to the placed computing device such that the user may be captured by the camera and images of the user displayed on the user interface displayed by computing device is visible to the user. (Act 304)

In various embodiments, if the physical environment surrounding the user is such that the user cannot comply with the Movement Conformance Engines prompts to reach a suitable or comfortable placement at a particular distance away from the computing device, the user may provide input data indicating the user is unable to comply with the Movement Conformance Engines prompts. In such a scenario, according to various embodiments, the Movement Conformance Engine may return to Act 302 in order to assist the user in setting the computing device in an alternate location such that the user may be subsequently able to comply with the prompts in Act 304. Similarly, the system may inform the user of other such issues that may interfere with recognition, for example, that the uses is are wearing clothing that is too dark, the environment is too poorly lit, etc.

The Movement Conformance Engine may display on the user interface on the computing device a series of key postures that are to be performed as part of an exercise(s) in order to guide the user through a correct and/or accurate performance of the exercise. (Act 306) During such guidance, the Movement Conformance Engine captures image data via the camera of the user performing (or attempting to perform) each key posture. As the user performs the key postures, the Movement Conformance Engine generates data representing various points and/or portions of the user's body with respect to conformant performance of the key postures in order to establish a baseline conformance range for the user. (Act 308)

According to various embodiments, for each key posture, the Movement Conformance Engine may display a guide armature that portrays a conformant performance of a key posture and may provide prompts and/or instructions requesting the user attempt to align a real-time image of their body displayed on the computing device with the displayed armature.

The Movement Conformance Engine sends video frames of the user's physical movements to a machine learning model that returns an incoming stream of armatures that correspond to the user and compares respective positions of the user's body joints and body portions represented in the user's armature with various armatures representing conformant performance of the key posture. In various embodiments, the user may provide voice input indicating a degree of effort and/or pain the user is experiencing at any given moment.

Upon capturing the user's baseline conformance range for the key postures, the Movement Conformance Engine guides the user to a starting position of an initial key posture of a first exercise. (Act 310) For example, the Movement Conformance Engine may provide audio and/or user interface prompts indicating the starting position (or starting key posture) that the user should attempt to perform. In some embodiments, the user may provide an input command, such as voice input data, indicating that the user has begun (or is ready to begin) the exercise(s).

The Movement Conformance Engine triggers initiation of the exercise(s) and displays an armature performing a series of the key postures. (Act 312) In various embodiments, the Movement Conformance Engine captures image data of the user's performance of the key postures and generates an armature of the user based on the user's real-time physical movements and may output various types of audio and/or graphic coaching prompt s based on real-time analysis of the joints and connection positions of the user's armature in comparison with a guide armature for a key posture(s). (Act 314) In various embodiments, the Movement Conformance Engine may execute one or more matching rules (further described herein) to generate output from comparison between the user's armature and the one or more guide armature for respective key postures.

Upon completion of a particular exercise, the Movement Conformance Engine may store results from the analysis of the user's armature via the matching rules. (Act 316). In some embodiments, the results (or a portion of the results) may be sent to a cloud computing platform for further analysis, such as, for example, analysis via one or more machine learning models. In some embodiments, a portion of the results may also be analyzed according to one or more pre-defined calculations locally executed on the computing device. The Movement Conformance Engine may further instruct the user to initiate another series of key postures for a subsequent exercise or the Movement Conformance Engine may terminate the exercise session. (Act 318).

In various embodiments, the Movement Conformance Engine captures physical parameters related to the user's armature over time and uploads the captured data to a processing and storage system. Immediate results from various types of analysis are communicated to the user, such as rep count, and other measures. Such immediate results may be determined by analysis and processing that occurs locally on the computing device.

In various embodiments, the video may be uploaded to a cloud computing system for further analysis as well. For example, the Movement Conformance Engine may upload the user's armature(s) and data representing armature changes from video frame-to-video frame. Various modules of the Movement Conformance Engine may be implemented on the cloud computing system to perform calculation with response to the armature changes, including (but not limited to): an amount of movement of each limb, a length of each ‘limb,’ and/or angular changes to each joint. Such angular changes may be, for example, angular velocity. Various types of calculated armature changes may further be represented as vectors and used by the Movement Conformance Engine within further machine learning models.

The Movement Conformance Engine may utilize a pool of data based at least in part on calculated anonymized armature changes from numerous users to determine standards for movement across various population segments. Such standards may be further based at least in part on coincident anonymized user data such as age, comorbidities, and conditions. From machine learning models and analytical methods, the Movement Conformance Engine may derive further changes to an exercise regime for any user as well as adjustments made to conformance range and/or rep count target of subsequent iterations of an exercise(s) and/or other exercises prescribed and other content of the system adjusted for the user. Additionally, the adjustments may include changes to the time a posture is held as in a stretch or in an exertion.

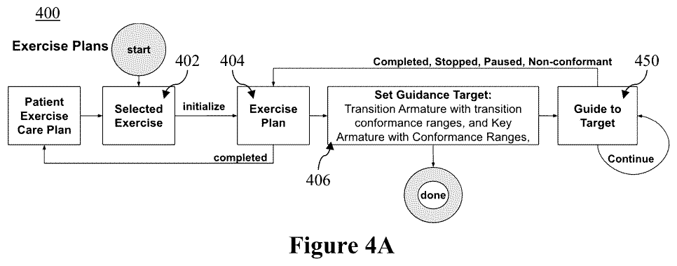

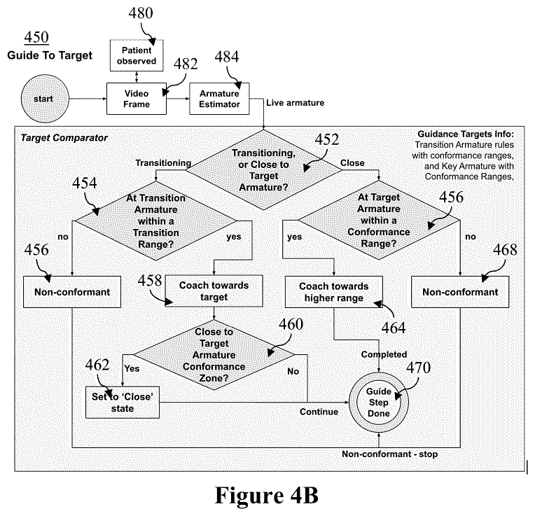

As shown in flowchart 400 of FIG. 4A, the Movement Conformance Engine may perform a guided exercise plan(s). The Movement Conformance Engine receives selections of one or more predefined exercises for a patient to perform as part of an exercise care plan. (Act 402). The Movement Conformance Engine initializes an exercise plan based in part on the selected exercise(s). (Act 404) An exercise care plan may describe an exercise(s) the patient should perform according to a certain number of repetitions during a pre-defined amount of time. In some embodiments, the exercise care plan may include multiple sessions of the exercise(s) at different times in order for the Movement Conformance Engine to capture data to determine a rate of health improvement of the patient.

As shown in flowchart 450 of FIGS. 4A and 4B, the Movement Conformance Engine receives an incoming stream of armatures representative of the user's various physical movements portrayed in video frames. The Movement Conformance Engine generates the video frames (Act 482) based on the camera of the computing device recording the user's physical movements. (Act 480). The Movement Conformance Engine feeds the video frames into a pose model which returns an incoming stream of data representative of the user's armatures and/or changes to one or more portions of the user's armatures. The Movement Conformance Engine utilizes the various instances of the user's armatures to determine whether the user's performance of the physical movements are conformant.

The Movement Conformance Engine guides the user in performing one or more physical movements such that the user's armature matches a target armature (or guide armature). The Movement Conformance Engine determines whether the user's armature matches (or is within a range close to matching) the target armature. (Act 452) The Movement Conformance Engine determines whether the user's armature represents that the user's performance is within a conformance range. (Act 456) If not, then the Movement Conformance Engine stops guiding the user towards the target armature. (Acts 468, 470) If within the conformance range, the Movement Conformance Engine provides coaching prompts to the user to motivate the user to improve their current performance in order to increase the measure of the user's conformance. (Act 464) The Movement Conformance Engine determines that the user's armature represents that the user has completed performance of a physical movement(s) and stops further guidance. (Act 470)

In addition, the Movement Conformance Engine confirms whether data related to transitions between instances of the user's armatures matches (or is within a range close to matching) an expected range of armature transition data. (Act 454) If the transition data is not within an expected transition range, the Movement Conformance Engine stops guiding the user towards the target armature. (Acts 456, 470) If the transition data is within the expected transition data range, the Movement Conformance Engine provides coaching prompts to the user to motivate the user to improve their current performance. (Act 458) The Movement Conformance Engine determines whether the user's armature represents a performance of the physical movement that is within a conformance range for the target armature. (Act 460) If so, the Movement Conformance Engine sets a close state (Act 462), sets a transition state of continue, and stops further transition guidance. (Act 470) The guidance step (Act 450) transitions with continue back to itself, whereupon the close state is utilized (Act 452, Paragraph #65 above)

The conformance can be provided as a binary result (e.g., pass/fail) or within a performance range which exceeds the binary result of having done enough. The range may be provided to the user so as to push the user to try for a higher level.

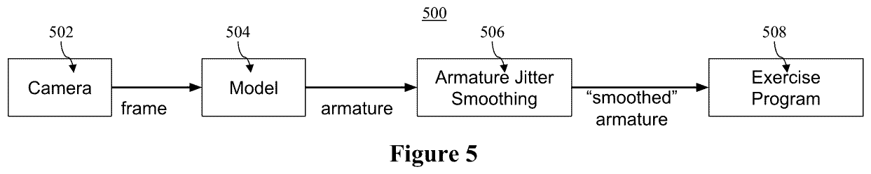

As the recognizer may operate on a single frame and obtain an armature for it, in a subsequent recognition it may obtain a slightly different set of locations for the key points and armature since the recognition process has some amount of jitter to it. Using a post-processing step to address ‘recognition jitter’ and smooth out the recognition. As shown in the flowchart 500 of FIG. 5, a camera(s) associated with the Movement Conformance Engine may continually capture image data, such as one or more image frames, of a user performing a predefined exercise(s). (Act 502) The Movement Conformance Engine may send as input the one or more image frames into a machine learning model in order to continually generate a skeletal armature representation of the user and various updates to the user's skeletal armature representation. (Act 504) As the Movement Conformance Engine generates data for rendering and displaying various instances of the skeletal armature representation of the user in the image data, the Movement Conformance Engine applies one or more averaging smoothing algorithms on one or more of the image frames. (Act 506) As a result of the averaging smoothing algorithms, the Movement Conformance Engine generates image frames with “smoothed” instances of the user's skeletal armature representation for display on a user interface as the user performs one or more movements of a predefined exercise during a set amount of time. (Act 508)

In various embodiments, the Movement Conformance Engine receives video frames generated by a computing device's camera(s) with image data portraying the user performing an exercise(s). The Movement Conformance Engine sends the received frames to an armature model module which returns an armature representing locations of body joints and connections of the user as they are portrayed in the video frames. The armature model may reside in a cloud computing environment, locally on the computing device or be distributed across the cloud computing environment and the computing device. In some embodiments, the armature model may be based on open-source software.

While the armature model can be in the cloud, it also can be on the device (e.g., a phone) so that it can operate in relative real-time when coaching the user. Additionally, whatever the means and speed of recognizing conformance the results can be delivered not only to the user but to other systems like the users health record, to the care management team, physical therapist, and to further systems for combining and assessing the aggregate data.

In various embodiments, the armature model may return subsequent armatures that correspond to the user from video frame to video frame whereby the received armatures may include recognition jitter. In some embodiments, recognition jitter results from the armature model returning armatures that are inaccurate and/or fail to represent consistent transitions of locations of particular joints and connections between successive armatures generated for a series of video frames. The Movement Conformance Engine implements a smoothing stage to the incoming stream of armatures received from the armature model in order to remove, correct, update and/or alter one or more respective armatures so as to remove recognition jitter.

In order to smooth the incoming stream of armatures, embodiments of the Movement Conformance Engine capture data from multiple armatures from the incoming stream and determines data averages for various body joints and/or connections over time. For example, the Movement Conformance Engine aggregates armatures that correspond to multiple video frames of the user performing an exercise(s) and applies a prioritization algorithm in order to give a different importance weight to various armature joints and/or connections in a set of armatures received during a particular window of time. The Movement Conformance Engine performs various types of averaging functions across the set of armatures to obtain successive armatures that include consistent and “smooth” location changes for joint and connections between successive armatures.

In other embodiments, various other approaches may be used. A “physical consistency” module may be used to take as input physical factors about the individual (e.g. limb length) and the previous/current pose. The module then outputs whether or not the current pose is valid. Alternatively, a “discriminator” may perform rejection sampling of poses at test-time. Furthermore, an another module may treat the 2D key points on the body as a 2D sequence, and then to measure distance between the correct/expected sequence of 2D key points and the predicted sequence. The module then returns a dense “score” on how close the user is to the desired motion.

Additionally, a module may take multiple frames as input (e.g., video models), and perform feature summation. Using the multiple frames, the module obtains aggregate features and then pass that to obtain the final prediction. The module can skip concatenating multiple frames worth of similar features to avoid expensive memory requirements.

In various embodiments, the smoothing stage implemented by the Movement Conformance Engine includes physiological correction filtering. Some recognition jitter may occur when a model swaps certain armature keypoints on the armature, such as swapping joint indicators that correspond to the left and right knees. For example, in a first frame, a left hip joint indicator of a first armature connects to a left knee joint indicator which in turn connects to left ankle joint indicator, and similarly for the right joints. However, a second armature, which corresponds to an adjacent successive video frame, identified by the model may include the left knee joint indicator where the right knee joint indicator should be—and the right knee joint indicator where the left knee joint indicator should be. Thus, the second armature may include one or more inaccuracies resulting from the swapped joint indicators. For example, a first inaccuracy may be that the left hip joint indicator in the second armature connects to the right knee joint indicator.

For physiological correction filtering, the Movement Conformance Engine defines a filtering window over a number (“N”) of video frames. For respective armature left keypoints (e.g., joint indicators for the left shoulder, left elbow, left wrist, left hip, left knee, left ankle) and respective armature right keypoints (e.g., joint indicators for the right shoulder, right elbow, right wrist, right hip, right knee, right ankle) whether their positions along an x-axis are to the left or to the right of the axis of symmetry for the body are tracked. In various embodiments, the positions may be based on joint indicator pixel positions given in the armature returned by the model. The Movement Conformance Engine defines a dominant orientation to represent a most common orientation with respect to the axis of symmetry over the last N frames (e.g., either left or right).

For a current video frame, the Movement Conformance Engine enforces the orientation given by the dominant position on the left and right keypoints of the corresponding armature returned by the model. For example, in some embodiments, the dominant orientation may indicate that left keypoints are to the right of the axis of symmetry and the right keypoints are to the left of the axis of symmetry. For example, the Movement Conformance Engine may detect that an armature corresponding to a current video frame portrays a left knee joint indicator to the left of the axis of symmetry, the Movement Conformance Engine swaps a position of the left knee joint indicator with a position of a right knee joint indicator in order to be in alignment with the dominant orientation.

In various embodiments, the smoothing stage implemented by the Movement Conformance Engine further includes moving average filtering. For any given video frame, the Movement Conformance Engine defines a smoothing window duration of time over N prior video frames. For every joint indicator in a current armature that corresponds with the given video frame, the Movement Conformance Engine adjusts the joint indicator's position based on its average position from the armatures for video frames within the smoothing window. In various embodiments, the Movement Conformance Engine may specifically execute moving average filtering with regards to video frames from the user's performance of a Sit-to-Stand exercise during an exercise time range of 30 seconds.

In various embodiments, the smoothing stage implemented by the Movement Conformance Engine further includes exponentially weighted smoothing. For any given video frame, the Movement Conformance Engine defines a smoothing window duration of time over N prior video frames. The Movement Conformance Engine further defines an exponentially decreasing set of weights during the smoothing window. Exponentially weighted smoothing prioritizes most recently viewed armatures over previously obtained armatures. For example, the Movement Conformance Engine may weight an oldest frame (in time) by e\hat{ }N, a second oldest frame by e\hat{ }(N−1) and so on, until a most recent frame (in time) is weighted by e\hat{ }1.

It is understood that any logarithmic or power relation can be applied and validated empirically. As such, the Movement Conformance Engine normalizes the various frame weights such that a sum of all the frame weights equals 1. To accomplish such normalization, the Movement Conformance Engine divides each weight by e\hat{ }N+e\hat{ }(N−1)+ . . . +e\hat{ }1. For each respective joint indicator in a current armature, the Movement Conformance Engine adjusts the respective joint indicator's position to a position determined according to a weighted average of that respective joint indicator's position in the various armatures within the smoothing window. In various embodiments, the Movement Conformance Engine may implement exponentially weighted smoothing for those types of exercise that generally requires fast movements, such as counting jumping jacks.

In various embodiments, the smoothing stage implemented by the Movement Conformance Engine further includes Savitzky—Golay (“Savgol”) smoothing. The Movement Conformance Engine defines a Savgol filter over the past N frames and applies the Savgol filter to an armature returned by the model that corresponds with a current video frame in order to smooth that armature. According to various embodiments, Savgol smoothing emits a frame with a certain amount of time lag (e.g., delay) having processed not only prior samples but subsequent ones.

Note that all smoothing algorithms require N frames to be captured within the smoothing window before smoothing can be used. In cases where frames do not have a fully specified armature due to noise, occlusion of the user, or other factors, the Movement Conformance Engine does not include those frames within the smoothing window, and instead only includes video frames that have corresponding fully specified armatures returned from the model.

Pose Recognition Jitter

As armature recognition can be noisy and erroneous, there can be a “recognition jitter” from frame to frame. This is a failure condition and it is dealt with in order to have a successful solution to recognizing poses in the real world.

This system takes video frames and passes them to a pose recognizer. This in turn is provided to a pose jitter recognizer. From there the video frames are passed for motion prediction.

The “Pose Jitter Recognizer” detects if there is jitter in the stream of pose recognitions received from the Pose Recognizer. If so, it will issue a jitter recognition signal. The Conformance Engine listens for the signal and uses it to guide the user, for example, to instruct them to dress differently or change locations in order to reduce the jitter.

Recognition failure conditions that give rise to jitter often relate to low-lighting, dark clothing, items in the background (such as paintings, pictures and/or other people), poor contrast between the person and the background, etc. Each of which can cause the pose recognizer to give a poor result. To deal with this, the system can be trained with more examples that can help to minimize this. The system can also be provided a filter that follows the recognition to identify mis-recognized armatures and issues a signal that the software can use to guide the user to improve the environmental conditions that lead to recognition problems.

Pose estimation networks can sometimes be prone to jitter in both the classification of a keypoint, as well as in the keypoint coordinates.

An example of classification jitter includes the swapping of classifications between keypoints that otherwise appear to be in the correct place. This can manifest in the swapping of left knee label with right knee label, or the left ear with right ear, etc.

Coordinate jitter can be measured in the amount of noise in the coordinate over time compared to the ground truth keypoint. For example, when keypoint coordinate jitter occurs, the recognizer, when given a video frame of a person standing, may provide the location of the left leg keypoint at a position x,y and then on the subsequent frame may recognize the location of the left leg as a,b even though the person has remained static.

To detect jitter and possibly remove erroneous armature detections, an instantiation could use any of the following solutions below:

Coordinate Jitter Correction
Low Pass Filters
   Rapid changes to motion from frame to frame (so long as the frame rate is fast enough and constant) are detected against a limit.
Biologically Informed Correction
   Some configurations of the body are achievable by a functioning body and some are not.
   The relative length of the limbs, and their relative angles can be used to detect unlikely body configurations.
   Changes to the body configuration at rates that are beyond normal human speeds can be an indication of the presence of jitter. For instance, if the left leg is on one side of the screen at one recognition and at the other side in the next recognition, then jitter is likely occurring.
Video Models
   Multiple frames can be passed into the pose estimation model to give more temporal information.
Non-Video Sensing
   As the armature sequence is the key intermediate for the rest of our downstream tasks, we can use other means than video processing and machine learning to gather this data. These options include wearable sensors and body scanning technology. Sensors have different accuracy characteristics and can be employed alone or together with video to detect poses.

Jitter Detection Via a Tracking Method

One method for correcting for classification jitter is to apply a tracking method. The method can apply to single pose or a sequence of poses.

A keypoint (or keypoint configuration) can be tracked from recognition to recognition, whether a classification or coordinate type, and if it exceeds a limit, then jitter is present. The system can either throw away the recognition or issue a jitter recognition signal.

Jitter presence can be aggregated in a variety of means and averaged (e.g., using a moving average). If a limit is exceeded, then the current recognition can be dropped, and if the aggregate jitter exceeds a limit (e.g., the jitter has gone on too long or too much), then the jitter recognition signal can be issued.

Correction of a Recognition in the Presence of Jitter

Biology can be used to inform how to correct for some of the coordinate jitter in a number of ways. Some of these ways include:
3D models can be used to represent the canonical human form (gendered or non-gendered). The system can deform or manipulate the 3D model in an attempt to match the predicted armature. Any discrepancy made between the deformed 3D model and the predicted armature can be propagated back to the predicted keypoints using a weighted average.
Utilize the measurements of each of the user's body parts to inform how to correct the coordinates. These measurements can be made manually or inferred through vision.

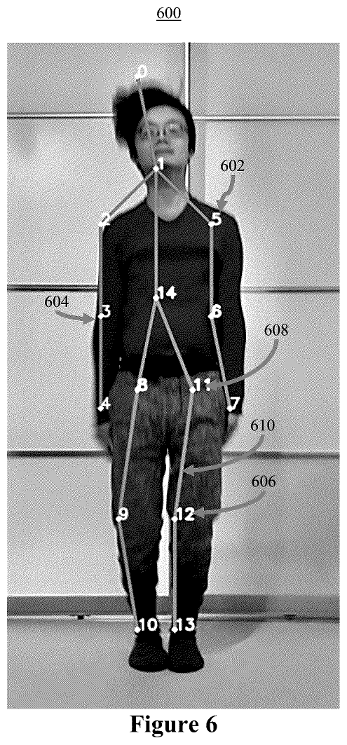

As shown in FIG. 6, the Movement Conformance Engine captures one or more images of a user's current position and stance. For example, a computer device with one or more cameras may be situated remotely from the user in order to capture image data of the user's current position and current stance and subsequent movements. According to various embodiments, the Movement Conformance Engine accesses an armature template that includes one or more joint indicators 602, 604, 606, 608. The Movement Conformance Engine utilizes pixel value data in the images of the user in order to determine where each respective joint indicator 602, 604, 606, 608 is to be displayed with respect to images of the user, which, in this example, is the landing position after a single ‘jumping jack’. In various embodiments, the Movement Conformance Engine may utilize two-dimensional (or three-dimensional) spatial coordinates in place of pixel value data.

The Movement Conformance Engine determines a placement for each respective joint indicator 602, 604, 606, 608, 610 for rendering a skeletal armature representation of the user and subsequent updates to the skeletal armature representation that correspond to the user's subsequent movements. As the user moves and changes a current position and current stance, the Movement Conformance Engine captures additional image data representing the user's movements and tracks changes in the pixel value data that corresponds to each respective joint indicator 602, 604, 606, 608, 610.

According to various embodiments, for example, if the user's left leg moves then the Movement Conformance Engine captures image data representing such movement. The captured image data may include pixel value data, such as a label of a joint indicator located at a particular pixel for the user's left knee as well as other portions of the user's body. For example, the pixel label value data for the user's left knee may be based on the color of the portion of the pants worn by the user that cover the user's left knee. The Movement Conformance Engine may associate the pixel label value and a first pixel region/location where the pixel label value is present in the image data with a left knee joint indicator 606.

According to various embodiments, the captured image data may further include movement of the user's right elbow. The captured image data may represent the pixel label value data for the right elbow as moving from a third pixel region/location to a fourth pixel region/location. As such, the Movement Conformance Engine concurrently updates the skeletal armature representation of the user such that right elbow joint indicator 604 is displayed with respect to the image data at the fourth pixel region/location.

It is understood that as the user's current position and current stance changes due to the user's physical movement, the Movement Conformance Engine continually (or continuously) captures image data and tracks movements of pixel label value data that correspond to the user's joints between various pixel region/locations. The Movement Conformance Engine therefore continually updates a placement and display of the armature joint indicators 602, 604, 606, 608, 610 that correspond with the movement of the pixel label value data. Moreover, a skeletal armature representation of the user may include any number of displayed connections between the respective armature joint indicators 602, 604, 606, 608, 610 and the Movement Conformance Engine may update a length of one or more of the displayed connections based on a changed distance between two joint indicators as measured between updated pixel region/locations. It is further understood that an armature template may have any number of joint indicators and any number of connections between joint indicator pairings. In various embodiments, the Movement Conformance Engine may have multiple different types of armature templates where each armature template is organized with anatomical point that more closely represent a type of movement, a type of exercise, or a stance that occurs during a particular exercise.

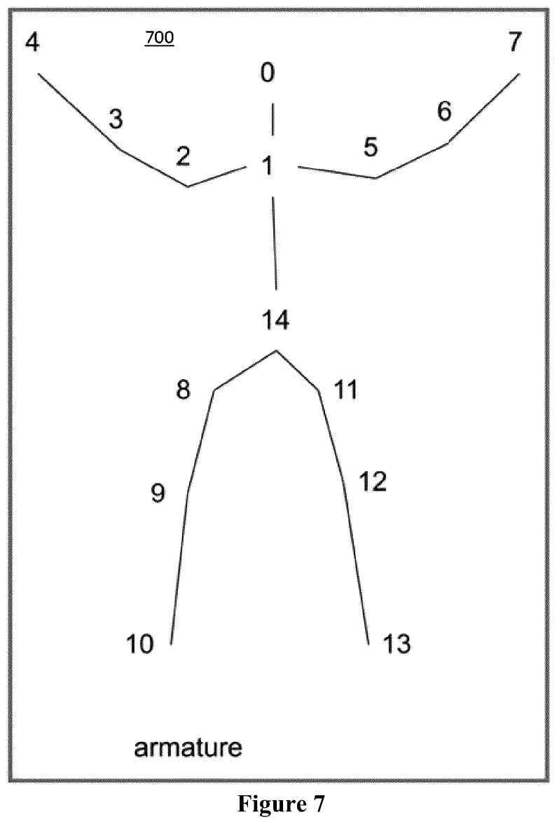

In some embodiments, an armature is a two-dimensional projection of keypoints of the underlying skeleton. For example, an armature may be a MPII-format 15-keypoint armature, where a particular keypoint indicates a position for the user's top of head, neck, left shoulder, left elbow, left wrist, right shoulder, right elbow, right wrist, left hip, left knee, left ankle, right hip, right knee, right ankle and the abdomen. As shown in FIG. 7, an armature 700 of the user may be based on an armature template that includes any number of joint indicators 0, 1, 2, 3, 4, 5, 6, 7, 8, 9, 10, 11, 12, 13, 14. In addition, the armature 700 includes displayed connections between joint indicator pairings. For example, a displayed connection may be a displayed line between two joint indicators 1, 2.

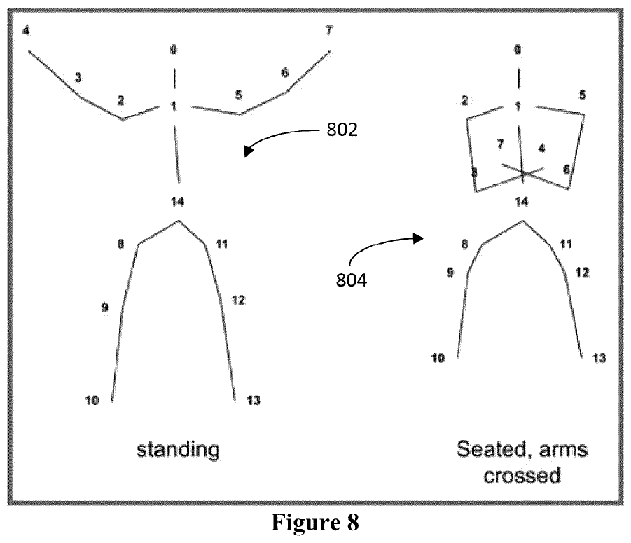

As shown in FIG. 8, the Movement Conformance Engine may have a first armature guide template 802 and a second armature guide template 804. The first armature guide template 802 may represent a beginning position/stance of a predefined exercise and the second armature guide template 804 may represent a terminating position/stance of the predefined exercise or a peak position/stance of the predefined exercise.

The Movement Conformance Engine may instruct a user to perform a predefined exercise where the user is initially positioned according to a position/stance represented by the first armature guide template 802 and attempts to transition to a position/stance represented by the second armature guide template 804 during a predefined time range. For example, the Movement Conformance Engine may concurrently display the user's skeletal armature and the first armature guide template 802 on a user interface. The user may physically move until the user may view joint indicators and displayed connections of the user's skeletal armature as being substantially aligned with the joint indicators and displayed connections of the first armature guide template 802. Once substantially aligned, the user may provide the Movement Conformance Engine with an input command indicating that the user has attempted to achieve the position/stance represented by the first armature guide template 802. For example, when the user can see that the user's skeletal armature is rendered on the user interface in substantial alignment with the first armature guide template 802, the user provides an input command to indicate that the user's current skeletal armature represents the user's performance of the position and stance of the first armature template 802.

As shown, ‘substantial alignment’ is automatically detected and that the amount of conformance is measured. Various parameters and thresholds may be used to provide a ‘close enough’ evaluation in the Engine. In some embodiments, a user can also call out to the system in order to request it make the conformance assessment. Such a call out can be done through voice, an interface or in time (e.g., after holding a pose for a sufficient amount of time).

In addition, according to various embodiments, the user may also physically move such that the user may view joint indicators and displayed connections of the user's skeletal armature as being substantially aligned with the joint indicators and displayed connections of a display of the second armature guide template 804. Once substantially aligned, the user may provide the Movement Conformance Engine with an input command indicating that the user has attempted to achieve the position/stance represented by the second armature template 804. In addition, the user may provide the Movement Conformance Engine with input indicating a degree of pain and/or difficulty the user is experiencing during a physical movement(s) and the Movement Conformance Engine may include data representative of such user-provided input into any of the determinations and calculations described herein.

The user's voice may be tracked by the Engine during armature tracking. Voice tracking can be assessed for expressions of ease or discomfort, e.g., “this is beginning to hurt”. The Engine may pause and/or initiate dialogue and motion tracking to enable a better description of the pain or movement limitation or to obtain further detailing of the experience with regard to a particle region of movement. The system may also repeat the pose in order to determine where the problem starts and/or ends, and obtain other language and particularization of the experience for further use in guidance and reporting of the state of the user.

Based on receipt of the respective input commands provided by the user, the Movement Conformance Engine captures pixel value data for joint indicators and corresponding lengths of connections of the user skeletal armature. For example, the input commands may be a physical gesture on a touchscreen of a mobile computing device and/or a voice command. Receipt of the input commands from the user indicate to the Movement Conformance Engine that the user has substantially performed a position/stance such that user's skeletal armature is currently displayed in substantial alignment with a displayed armature guide template 802, 804. The Movement Conformance Engine records the placement of the joint indicators, the connections and the connection lengths for the user's skeletal armature as reference points to be used to measure whether the user can perform a predefined exercise in conformance with an expected measure of competency (or conformance).

According to various embodiments, reference points may also be used to estimate how the user's skeletal armature is expected to appear when the user has completed a predefined exercise. For example, if the predefined exercise requires the user to start at the standing position and then end at a seated position, the Movement Conformance Engine may further capture reference points for the user's skeletal armature based on image data portraying the user in a seated position. In various embodiments, the user may cycle through repetitions of a predefined exercise during a set amount of time. Each time the Movement Conformance Engine generates a current version of the user's skeletal armature that aligns with the seated position reference points the Movement Conformance Engine may further count the occurrence of such alignment as an indication that the user has completed a cycle (e.g., rep count) of the predefined exercise.

According to various embodiments, the Movement Conformance Engine may have access to a set of matching rules that correspond with a predefined exercise(s). For example, the matching rules may represent a plurality of expected armature templates based on positions/stances that occur according to a specific sequence during a correct performance of the predefined exercise(s). The matching rules thereby include data describing armature change relationships between changes in placement of the joint indicators, changes in placements of joint indicator connections and amounts of acceptable changes and angle between connection lengths as well as changes in angles between connections.

Optionally, the system may use an ordering where the sequence of exercises and loops of movement can be specified and identified so long as they are performed in sequence (e.g, the pose conforms to the specification). In other embodiments, the system may use ‘unordered movement’ where the user performs whatever movements they wish, and the Engine recognizes a specified posture from a set of specifications should the specified pose be performed.

According to various embodiments, upon capturing the reference points for the user's skeletal armature, the Movement Conformance Engine may prompt the user to begin performance of a predefined exercise within a set time range. For example, the Movement Conformance Engine may prompt the user to attempt to complete five counts (or reps) of a particular exercise within the set time range. As the user performs the predefined exercise, the Movement Conformance Engine continually analyzes image data of the user's movement to update the joint indicators, connections and connection lengths of the user's skeletal armature. The Movement Conformance Engine continually compares the user's current skeletal armature to the matching rules in order to determine a measure (or degree) of conformity of the user's performance with threshold performance of the predefined exercise as represented by the armature change relationships in the matching rules. In some embodiments, the matching rules may further include rules for transitions between armatures. For example, the matching rule may describe changes that occur during transition from one armature to another armature, where such changes are indicative (or not indicative) of conformance.

For example, the matching rules may indicate that a conforming performance of the predefined exercise may result in a range of acceptable amount of changes of joint indicator locations and acceptable amount of changes to connection lengths between joint indicator pairs as the user's skeletal armature takes on various positions and stances that correspond to the armature change relationships represented by the matching rules. Note that these lengths may correspond to body size and morphology, but they may also refer to the angles between the joints. The lengths (as well as angles) may change over long time frames (e.g., as the user ages) as well as change due to the motion in real time as projected to a 2D projection of the 3D world. The Movement Conformance Engine compares the user's skeletal armature to the matching rules' armature change relationships in order to determine a degree of conformity between the user's performance.

According to various embodiments, the matching rules may be implemented according to a machine learning network trained on training data representing various segments of individuals performing various predefined exercises. For example, various segments of individuals may include patients within a certain age range, from a particular geographic location, with similar physical limitations, with similar medical problems and/or similar rates of health improvements (e.g., mobility improvements). As such, the Movement Conformance Engine may generate input data based on the user's skeletal armature and feed the input data into a machine learning network. Output from the machine learning network may represent the user's degree of conformity. In various embodiments, the user may select and/or change which segment(s) of individuals the user wishes to be compared against. In addition, output from the machine learning network may include diagnostic output indicating a confirmation of a certain degree of shaking experienced by the user during performance of an exercise whereby such output indicates a likelihood of Parkinson's disease.

According to various embodiments, a user may stand in place while holding a smartphone in the user's right hand. The user extends the right arm while keeping the right arm straight. The user performs a circular motion (or arc motion) with the straight extended right arm while minimizing movement of the user's body and/or torso. The Movement Conformance Engine determines a distance from the right shoulder to the smartphone based on a radius of a theoretical sphere being recorded by the smartphone's motion sensor during the circular motion. As such, the Movement Conformance Engine identifies a position of the right shoulder in three-dimensional space and may utilize the identified position to determine visual placement of a right shoulder joint indicator for the user's skeletal armature.

The Movement Conformance Engine may further instruct the user to hold the smartphone with the right hand bent. Placement of the smartphone as a result of being held by the bent right hand allows for the Movement Conformance Engine to further determine a distance between the right shoulder and the right wrist. Based on the distance between the right shoulder and the right wrist, the Movement Conformance Engine identifies a position of the right wrist in three-dimensional space and may utilize the identified position to determine a visual placement of a right wrist joint indicator for the user's skeletal armature. It is understood the determining a radius with respect to circular motion (or any motion that includes four non-colinear points) of a particular extended body part with respect to the user's still body or torso may be utilized to determine a position in three-dimensional space for a joint indicator for any type of body part on the user's body.

In various embodiments, the Movement Conformance Engine may initiate tracking of circular motion by initially executing a three-dimensional (3D) setup. The Movement Conformance Engine may instruct the user to scan the user's surrounding environment. During the scan, the Movement Conformance Engine captures camera and sensor (e.g., gyroscope, accelerometer) data via the computing device to confirm and/or establish 3D coordinates for a location and/or position of the computing device.

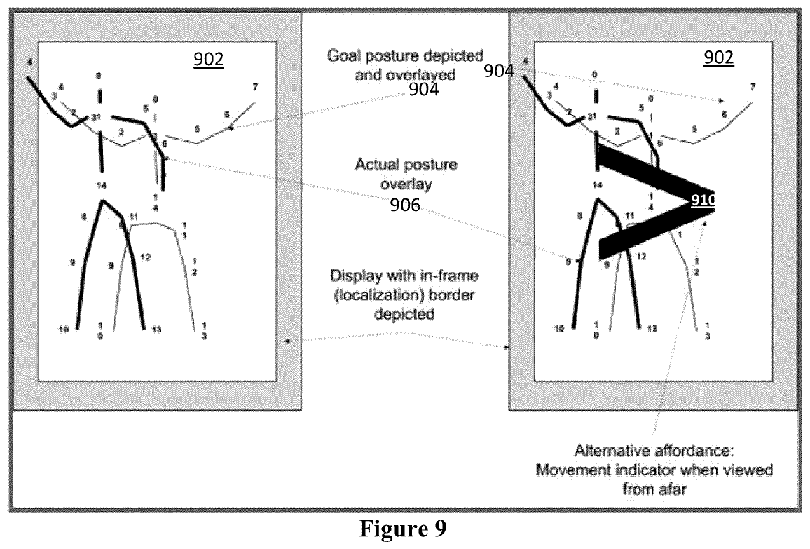

As shown in FIG. 9, the Movement Conformance Engine displays a user interface 902 that includes display of a guide armature 904 and an armature 906 representing a current position and stance of the user. The Movement Conformance Engine displays one or more movement indicators 910 in order to prompt the user to move the user's current position and stance such that the armature 906 rendered by the Movement Conformance Engine and displayed in the user interface 902 may be rendered in alignment with—and overlaid upon—the guide armature 904.

According to various embodiments, aspects illustrated in FIG. 9 may further be utilized for guiding a user into an initial key posture at the outset of performance of an exercise(s). In such a scenario, the Movement Conformance Engine confirms whether an image of the user portrays the user as being completely in frame. In various embodiments, the Movement Conformance Engine may display on the border of the user interface to indicate when the user is in frame in a centered position in the user interface. The Movement Conformance Engine may further modify the color of the border to indicate additional conditions. A certain colored border may be used to indicate, for example, whether the user is position too far away from the camera.

In some embodiments, an armature can be recognized but also a silhouette of the person can be recognized as an indication of shape (body morphology). The shape can be used to adjust matching rules, for example, where a person's physical shape prevents the arms from being extended downward fully to vertical. The shape of limbs and body can be used to adjust the matching rules for a given body shape so that a matching such as ‘arms down as much as possible’ takes into account that the arms may not be vertical. Thus, the Engine can determine that a target posture is limited by body morphology not only by limitations of the range of motion of a given joint.

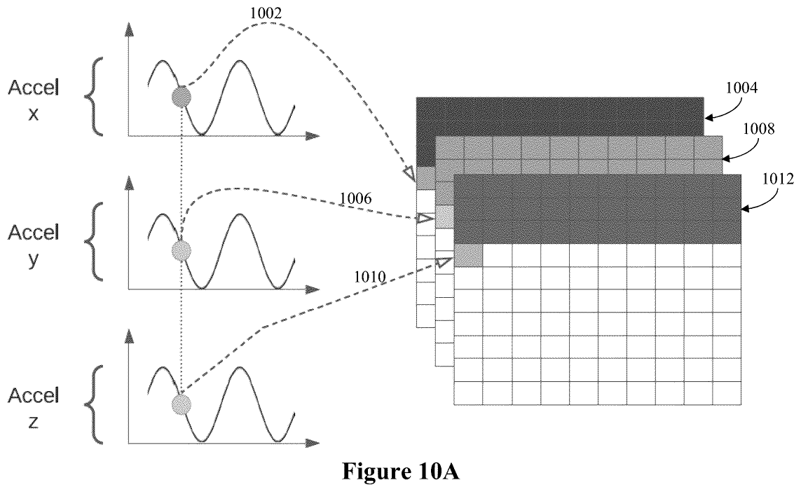

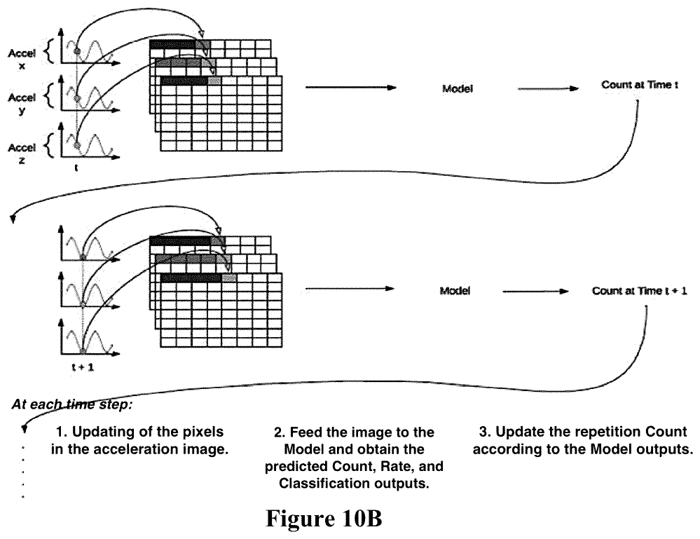

As shown in FIG. 10A, the Movement Conformance Engine converts movement data, such as sensor data, into image data and generates machine learning input data based on the image data. The Movement Conformance Engine feeds the machine learning input data (based on the image date) into one or more machine learning models and machine learning output indicates the user's exercise rep counts and various statistical measures of the user's performance (e.g., conformance, comparisons).

According to various embodiments, a computing device may be held by the user or attached to the user's body while the user performs one or more predefined exercises during a set amount of time. The Movement Conformance Engine captures sensor data from a motion sensor of the computing device. For example, the Movement Conformance Engine captures accelerometer data from one or more accelerometers of the computing device. For example, the sensor data may represent the user's movement according to acceleration values 1002, 1006, 1010 in a three-dimensional space.

The Movement Conformance Engine converts the positional coordinates 1002, 1006, 1010 into image data and further inputs the converted image data into a machine learning network. For example, the various values of the acceleration values 1002, 1006, 1010 may be converted into RGB (red, green, blue) image data. According to various embodiments, each axis (x, y, z) for the positional coordinates 1002, 1006, 1010 may respectively be represented by a specific color. For example, the x axis may be represented by the color red. The y axis may be represented by the color green. The z axis may be represented by the color blue.

In various embodiments, each positional coordinate 1002, 1006, 1010 is a value that reflects a change in velocity (e.g., acceleration). For example, the Movement Conformance Engine captures accelerometer data and the Movement Conformance Engine builds an image based on three colors (red, blue, green) that each correspond to a particular x, y, z dimension of the recorded velocity change at a given point in time. Each positional coordinate 1002, 1006, 1010 thereby has a changing value that corresponds to the change in velocity detected by the motion sensor. For example, velocity change coordinate values may fall within the range of 0 to 255. A first value for the z velocity change coordinate 1010 at first moment during the user's movement may be 200 and a second value for the z velocity change coordinate 1010 at a second moment during the user's movement may be 255. Since the color blue corresponds to the z velocity change coordinate 1010, the first value (200) for the z velocity change coordinate 1010 maps to a lighter shade of blue than the second value (255). Values for the x velocity change coordinate 1002 also fall within the range of 0 to 255 but map to varying shades of the color red. Values for they velocity change coordinate 1008 also fall within the range of 0 to 255 but map to varying shades of the color green.

The Movement Conformance Engine builds an image 1004, 1008, 1012 based on pixels that reflect the normalized acceleration coordinates (x, y, z) 1002, 1006, 1010. Each moment during the user's movement that is represented by the sensor data will have corresponding (x, y, z) velocity change coordinate values that map to respective shades of red, green and blue. Each particular moment during the user's movement will correspond to a particular pixel of the image. The Movement Conformance Engine inserts the shades of red, green and blue for the (x, y, z) velocity change coordinate values from a particular moment during the user's movement into the same pixel of the image. For example, if the motion sensor generates motion data during a moment of stillness that occurs during the user's performance of a predefined exercise, then the (x, y, z) velocity change coordinate values for the still moment would be (128, 128, 128). The Movement Conformance Engine determines the shades of red, green and blue that maps to the value of 128. In such a case, all shades of red, green and blue would be grey. As such, the Movement Conformance Engine inserts white into a pixel that corresponds to combined color shades for the (x, y, z) velocity change coordinate values from the still moment during the user's performance of a predefined exercise.

Therefore, an entire image(s) built by the Movement Conformance Engine can be based on shades of red, blue and green to represent an entire performance of a predefined exercise by the user, or an entire movement performed by the user. According to various embodiments, an image built by the Movement Conformance Engine may be rectangular-shaped image with 1800 RGB pixels. The Movement Conformance Engine feeds the converted image data into a machine learning network trained on image data in order to generate output that indicates various attributes and characteristics of the user's movement(s).

According to various embodiments, the user may be holding the computing device in the user's hand during performance of the predefined exercise. The Movement Conformance Engine may receive input data from a touchscreen of the computing device during performance of the predefined exercise and the Movement Conformance Engine may determine a correlation between the received touchscreen input data (or voice input data) with the position and/or position change data. For example, the user may perform various swipe gestures on the touchscreen while performing the predefined exercise. Various types of swipe gestures may be predefined as representing various degrees of difficulty and/or pain currently experienced by the user during performance of the predefined exercise.

The Movement Conformance Engine captures the input data based on the user's swipe gestures and associates the swipe gesture input data with the corresponding (x, y, z) velocity change coordinate values generated by the motion sensor when the swipe gesture input data was received. The Movement Conformance Engine may further generate user interface data to generate a display of a graph presenting changes and relationships between the various degrees of difficulty and/or pain experienced by the user and various periods of conformity or lack of conformity of the user's performance of the predefined exercise. The Movement Conformance Engine may further identify particular time ranges that occurred during the user's performance of the predefined exercise where a threshold degree of conformity regressed to a lack of conformity and may further determine one or more muscle groups being used in the predefined exercise during the identified particular time ranges.

As shown in FIG. 10B, a motion sensor (such as an accelerometer) of a computing device may generate sensor data while a user performs a predefined exercise(s) during a set amount of time. For example, the user may be holding the computing device during performance of the predefined exercise. The motion sensor generates sensor data that the Movement Conformance Engine converts to three-dimensional changes to velocity (x1, y1, z1), (x2, y2, z2), (x3, y3, z3) . . . that represent the computing device's acceleration at a given moment of time during performance of the predefined exercise(s). For example, each respective sensor data may correspond to one second or one millisecond.

The Movement Conformance Engine converts each velocity change coordinate value x1, y1, z1, x2, y2, z2, x3, y3, z3 . . . from a normalizing acceleration RGB value range from 0-255. As illustrated in FIG. 10B, the Movement Conformance Engine builds the image by inserting one pixel at a time, for each respective unit of measurement, according to chronological order. In some embodiments, the image is initialized with (128,128,128) (e.g., velocity change coordinated values that are representative of no movement), and then at time unit 0 the first pixel is added, then at time unit 1, the next pixel is added, and so forth.

In some embodiments, the image space may be large enough that an entire instance of a recorded motion could fill the image space multiple times. In such cases, a pixel for each respective time unit is doubled or tripled. That is, each pixel is inserted into the image by the Movement Conformance Engine twice or three times. For example, video from a 30 second duration that portrays performance of an exercise, sampled at 60 hz, results in an image built with 1800 pixels. In some embodiments, in order to train a machine learning network, such an image provides 1800 different pixel examples to be used as training data.

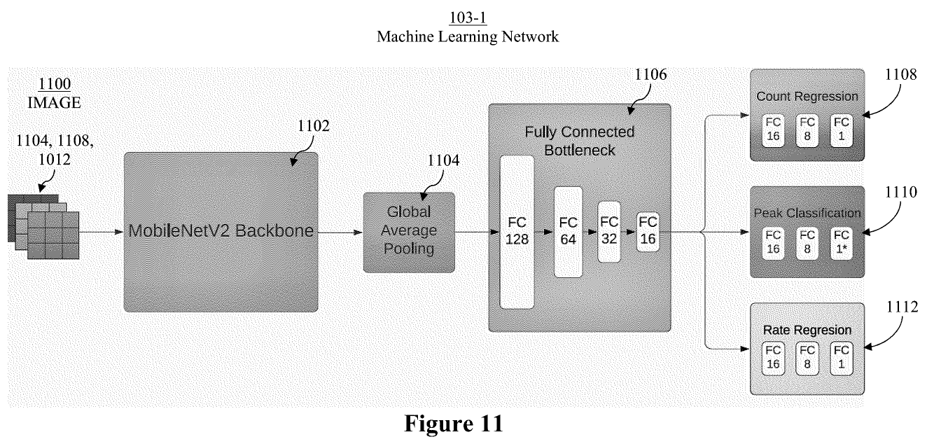

As shown in FIG. 11, the Movement Conformance Engine inserts input into a machine learning network 130-1 trained on various types of image training data. The image training data may be representative of various performances of various types of predefined exercises from one or more individuals that belong to one or more individual segments. The input may be based on image data 1100 built by the Movement Conformance Engine as a result of converting motion sensor data into pixel values representing various shades of red, green and blue. According to various embodiments, the machine learning network 130-1 may include a MobileNetV2 Backbone module 1102, a Global Average Pooling module 1104, a Fully Connected Bottleneck module 1106, a Count Progression module 1108, a Peak Classification module 1110 and a Rate Progression module 1112. According to various embodiments, the Count Progression module 1108 may be directed to detecting how many instances or repetitions of a particular predefined exercise performed by the user are represented in the image 1100. The Peak Classification module 1108 may be directed to detecting image data that represents a peak of each instance a particular predefined exercise performed by the user. The Rate Progression module 1112 may be directed to detecting image data that represents a rate at which the user is able to repeat respective cycles or counts of the predefined exercise.

For example, if the predefined exercise requires the user to begin at a seated position and perform movements to arrive at a standing position and then ultimately return to the seated position, the Count Progression module 1108 detects respective instances (e.g., reps, counts) in the image data 1100 that represent the user moving from a seated position through the standing position and back to the seated position. The Peak Classification module 1108 detects image data representing a peak within each count. For example, a peak may be represented by the user reaching the standing position and beginning a descent back to the seated position.

Various embodiments of the Movement Conformance Engine may use any suitable machine learning training techniques to train the machine learning network 130 for each sensor, including, but not limited to a neural net based algorithm, such as Artificial Neural Network, Deep Learning; a robust linear regression algorithm, such as Random Sample Consensus, Huber Regression, or Theil-Sen Estimator; a kernel based approach like a Support Vector Machine and Kernel Ridge Regression; a tree-based algorithm, such as Classification and Regression Tree, Random Forest, Extra Tree, Gradient Boost Machine, or Alternating Model Tree; Naïve Bayes Classifier; and other suitable machine learning algorithms. In some embodiments, multiple types of machine learning models may be used for a particular time range of an exercise(s).

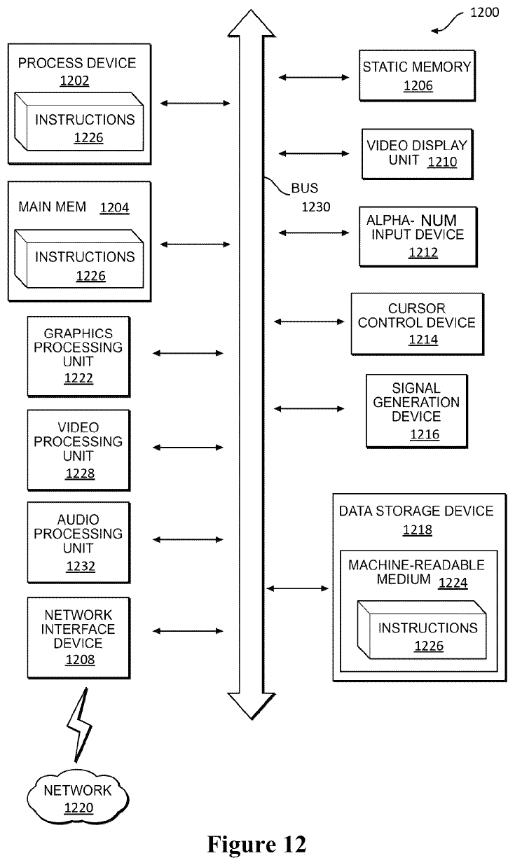

FIG. 12 illustrates an example machine of a computer system within which a set of instructions, for causing the machine to perform any one or more of the methodologies discussed herein, may be executed. In alternative implementations, the machine may be connected (e.g., networked) to other machines in a LAN, an intranet, an extranet, and/or the Internet. The machine may operate in the capacity of a server or a client machine in client-server network environment, as a peer machine in a peer-to-peer (or distributed) network environment, or as a server or a client machine in a cloud computing infrastructure or environment.

The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 1200 includes a processing device 1202, a main memory 1204 (e.g., read-only memory (ROM), flash memory, dynamic random access memory (DRAM) such as synchronous DRAM (SDRAM) or Rambus DRAM (RDRAM), etc.), a static memory 1206 (e.g., flash memory, static random access memory (SRAM), etc.), and a data storage device 1218, which communicate with each other via a bus 1230.

Processing device 1202 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 1202 may also be one or more special-purpose processing devices such as an application specific integrated circuit (ASIC), a field programmable gate array (FPGA), a digital signal processor (DSP), network processor, or the like. The processing device 1202 is configured to execute instructions 1226 for performing the operations and steps discussed herein.

The computer system 1200 may further include a network interface device 1208 to communicate over the network 1220. The computer system 1200 also may include a video display unit 1210 (e.g., a liquid crystal display (LCD) or a cathode ray tube (CRT)), an alphanumeric input device 1212 (e.g., a keyboard), a cursor control device 1214 (e.g., a mouse), a graphics processing unit 1222, a signal generation device 1216 (e.g., a speaker), graphics processing unit 1222, video processing unit 1228, and audio processing unit 1232.

The data storage device 1218 may include a machine-readable storage medium 1224 (also known as a computer-readable medium) on which is stored one or more sets of instructions or software 1226 embodying any one or more of the methodologies or functions described herein. The instructions 1226 may also reside, completely or at least partially, within the main memory 1204 and/or within the processing device 1202 during execution thereof by the computer system 1200, the main memory 1204 and the processing device 1202 also constituting machine-readable storage media.

In one implementation, the instructions 1226 include instructions to implement functionality corresponding to the components of a device to perform the disclosure herein. While the machine-readable storage medium 1224 is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media.

Various embodiments provide an exercise language or movement description, and how coachable exercises are authored using it. The language can be used by a programmer to build an exercise, however, an ‘authoring tool’ may be used by non-programmers to craft exercises as well. In addition, movement descriptions, defined by a language or using a visual tool, can be used to organize the component parts of moment so they can be aggregated into larger chains of movement to generate real-world, complex exercises and exercise routines. Additionally, a library of reusable movement components may be constructed to assist generating such exercises.

The exercises and routines can be parameterized to capture the particulars used for individual users or patients to be safely guided in preventative and recuperative movement. The system may also report on conformant moment performance to the user as well as more in-depth biologically specific information to medical practitioners and administrators. The visual affordances (means) may enable editing by non-programmers borrowing metaphors from other systems (e.g., cartoons, and personal video editing).

Pose Recognition with an Exercise Definition Language

A coach module/program/application can guide a user by generating speech, by presenting images and text on a screen (e.g., a smartphone), and by listening to the patient. It incorporates speech generation and speech synthesis together with the ability to recognize motion in real-time to enable a dialog with the patient as they move. The Coach guides a patient through motion and responds to a patient's voice and behavior.

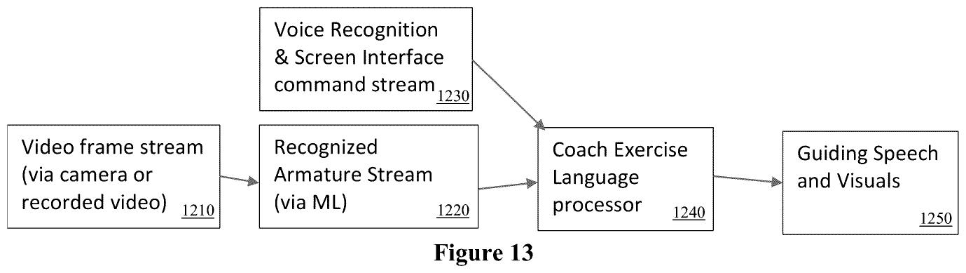

As shown in FIG. 13, the coach technology takes as input a video stream 1210 and interface commands (voice, and touch), then passes the video stream to a human-pose recognizer that emits in real-time a skeletal representation of the body, if a human body is present 1220. The stream of recognized skeletons (or armatures) is processed in real time against a movement and coaching representation. That representation can be thought of as a language. The Coach application can load a representation of an exercise expressed in that language and compare the determined armatures to the desired representation 1240 to determine the differences between the stream of armatures and the desired representation of motion and guide the user to perform the desired motion based on differences between the actual and desired armatures 1250. This can be run in a smartphone or personal computer. The device and software also present the stream of interface actions done by the user like pressing button, touching the screen, and moving a pointer, along with a voice recognition stream to supply user driven commands to the Language processor to interact with coach alongside of the user's larger physical movement 1230.

Note that this process can be iterative, where guidance may be given and additional video is recorded for recognition. Likewise, in some embodiments, the coaching technology may begin with speech and visuals being provided by the exercise author.

Exercise Description Language

A hierarchical motion representation includes constraints, steps, exercises/motions, and routines. The hierarchy goes from minute definitions of position in a time-instant and organizes them into an expected ‘step’, as one might describe dance. The steps are then organized into sequences that comprise a describable motion or exercise. These motions or exercises can then be organized into routines.

In various embodiments, the term ‘language’ is a computable, parseble language that a machine can execute. Accordingly, it can be a programming language or computable representation. It some embodiments, it can be text that can ‘read’ by a language model. The language is able to describe movement in moment-by-moment detail such that the movement is measurable and conformant to a specification.

Specific motions, like ‘getting into frame’ (e.g., telling the person to get the various parts of their body visible to the camera so that coaching can be done), are treated as reusable modules and can be shared when putting together a description of more extended or complex motion. By chaining together such component motions, one can script an extended exercise. For instance, “get the shoulders and head into frame” and then “count head tilts”. Each of the items can be referred to as exercises or movements that include one or more steps (and the step's constraints) that encompass that movement. Each of the component steps have movement constraints that define a desired target pose as well other non-target postures with coachable responses to them. For example, to get a person's head and shoulder visible for a head-tilt exercise, the motion step may have a ‘target’ of seeing their head and neck and shoulders, and have a set of ‘warnings’ for the head not being visible or their shoulder not being visible with a coaching response of “please get your head into view” and “please bring your shoulders into view”, respectively.

An exercise can also loop steps akin to ‘for loop’ in software: loop “tilt head to left” and “tilt head to right” for 5 reps. Such a loop can be defined as repeated steps, for example, a “tilt head left” followed by a second step, “tilt head right”, to be done until some condition is met, e.g., 5 loops are done, and/or some amount time has elapsed, etc.

In turn, a Routine can be pieced together by chaining together pre-built exercises with some interstitial exercises to form a coherent coaching experience for the user.

Each component, from constraints, to steps, to movements, to routines are named and reusable. The specifics are parameterized, so that an actual desired count for an exercise is supplied by the enclosing context. In other words, a jumping jack exercise can be used provided to one patient with a 10-rep count, and for another with a 20-rep count.

Parameterized Motion

Physical therapy type parameters like rep counts, time limits, and degrees of motion can be left up to the specifics of the therapist and the patient. Similarly, aesthetic parameters like counting upward versus counting downward to zero, or the speed of the counting are also parameterizable.

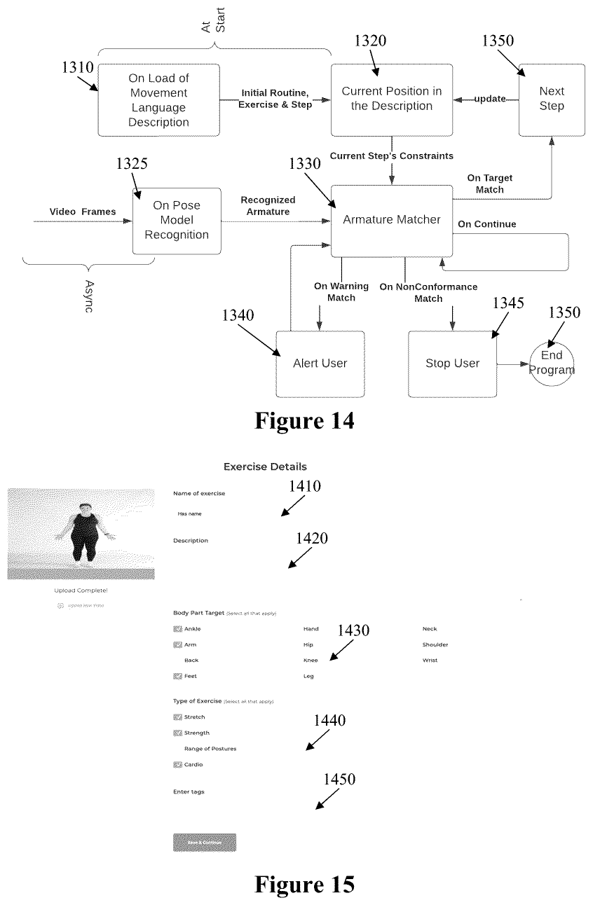

FIG. 14 shows a process flow for a step matching method. At the start of a routine, the coach application loads the Movement Description for that routine 1310. The Movement Description (sometimes referred to as language) contains the components of the exercises that make up the routine, and in turn, the movements that comprise each exercise. Each of these can be separately loaded components that can be independently loaded and shared with the Descriptions of other routines. Within the exercises are steps that contain constraints. A step's constraints may be grouped into those constraints that identify the movement to be achieved, the movement that the coach should communicate to the user about (e.g., warn), and movement that is undesired or unsafe (e.g., non-conformant).

FIG. 14 shows the loading of the Description (language) for the position at the start 1320, how video frames are analyzed to determine a Pose Model 1325 and are asynchronously recognized as ‘armatures’ by the mode 1330, and, on armature recognition, are processed with the current step of Movement description to take some action. The possible actions include alert the user 1340, move to the next step 1350 in the Movement description, or stop if the user is doing an unsafe motion 1345.

The Movement Description is similar to a programming language, with a current position (like a program counter), where the current position is updated when the current step's target constraints are matched and the current position moves to the next position. The program runs to the end and then stops. Various steps in the program may initiate interactions with the user. The program can be interrupted by the user as well through asynchronous commands.

Within a ‘step’ are the underlying constraints for the various sets of expected postures. A posture constraint can define a pose that is targeted for the user to achieve, a pose that causes warnings to the user, or a pose that is unsafe so as to necessitate safely securing the user and ending the activity. In further embodiments, there can be other definitions for poses as well such as a higher or lower performance class (e.g., holding the leg at a higher or a lesser range of motion, or velocity, etc.).

Each pose has a set of constraints, matching rules, and effects on the Coach. The target posture includes constraints the patient is to match. On a match, the step is no-longer active and enclosing Movement (Exercise) is notified so it may locate the next step. The constraints may define how the patient is to hold themselves (e.g., angles of arms, etc.), and/or how long they are to hold themselves in the pose. Thus, a “pose’ includes a set of constraints (or even a single constraint) that can create some response by the coach. These can be in the form of matching rules with respect to a video and detected armature in real time.

A warning posture provides a list of constraints which, when matched, cause a corresponding and specified response to the user. (e.g., the coach application may speak to the user explaining a corrective action, such as, “raise your arm higher”).

A non-conformant posture is a set of constraints, which, if any match, the user is asked to stop and the routine is ended.

In one, non-limiting implementation, any number of posture types can be created, and the ordering of the set of constraints and the matching rules for the set can be specified (e.g., match in order and all must match). The actions to be taken on matching are spelled out. In some implementations, the three types are identified and limited as above for implementation convenience.

In one, non-limiting implementation, certain movements may cause branching to other steps, and in this way a behavior driven interface can be achieved. This can allow the user to perform a gesture that might have the coach speak louder. In an alternative implementation, an additional set of constraints can be added that detect a gesture and use it similarly to control the coach or exercise experience by the user. This may be in addition to the ability of the coach to be controlled through speech and voice commands, e.g., “speak louder”. Some commands may control the flow of the routine or exercise, for example, the user may say “I want to do another rep”, “stop”, “get me another routine for my back”, etc.

Counted Exercise

In one, non-limiting implementation, an exercise can loop over the steps until some limit is hit either in time and/or in loop-count. In this way, rep and time counted exercises are implemented. When the loop occurs, the counts are increased and spoken to the user. The constraints can control what is spoken or displayed to the user to represent the counting. In another non-limiting implementation, an exercise can be paced by music at a certain beat count, including rhythmic exercises start counts and position changes such as “now your left in 3, 2, 1 . . . ”.

Static Positions and Stretches

The ‘steps’ are built from a set of ordered constraints on a given armature. If all the constraints match, then a ‘step’ is recognized. When the step is recognized then a number of coaching actions are possible, such as, counting (e.g., when in position for a jumping jack), or time is counted. Using an interface, an author may easily specify the coach application's behavior that corresponds to the current state of the exercise program. Some steps are held, like during a stretch, so time is counted. A step may be completed instantaneously or after a period of time. A step may have a time constraint for it to complete, or it may simply be a time delay used to pace an exercise.

When describing a constraint, the author can specify various features. The system can count and pace movements as desired. The style of rep counting is selectable, such as, counting reps upward to a limit or downward toward zero. Likewise, the style of time counting can be either: counting down to zero or counting to a time limit. The coach program can be set to skip counts (e.g., every other number) instead of monotonic counting. Similarly, the pacing for timing can be set to go by the second or some other period (such as, beats in a rhythm).

When a position is held, as in in a stretch, then a step defines parameters to control how long the user is to hold the position until the target position is recognized, or to count how long the current target is being held and to recognize that the target is achieved after the position is no-longer recognized (e.g., to recognize the position as being on-target but does not complete the recognition until the user stops holding the position and recording the time the position is held on target.). These enable the holding of a static position either for a desired time or for a time that is as long as possible by the user.

In one, non-limiting example, a jumping jack could be a loop of a first position that is standing with legs together and arms down, and then a second position that is legs apart and arms up. The constraints provide means to decide how close the legs must be together when standing, and how far apart after jumping, as well as the position of the arms. This set of constraints for a step are required to match in order for a step to be completed. The steps can also provide a set of constraints to warn the user, and another set of constraints to stop the exercise entirely (e.g., if the armature indicates the user is no longer standing (in case they've fallen), the exercise would be stopped).

Movement Reporting in the Language

The Coach makes various measures of the body moment by moment (e.g., speed of limb movement, range of motion of a joint, the number reps performed, etc.). The language affords describing which constraints, in which step of an exercise are required to be measured and reported on. The language describes how (or if) the information is reported to the user and separately reported to the system as part of the patient's history.

The system is provided details for specifying or selecting the results that matter for saving and for display. The program can measure multiple parameters depending on the constraints, and from these a subset is made available as presets for selection to save for physical therapist, doctor, or other healthcare service provider use and/or for display to users. A markup language can be used to refer to a step, its constraints, and/or to the results found. As an example, the results may be the maximum or minimum value or a rate of change, an average over a period of time, first and last values, etc.

Library of Routines, Exercises and Components of Motion.

A library of motions at each level of movement detail can be built by directly using the exercise description language itself (e.g., as text) or via visual affordances that present selectable, prebuilt units of movement that can be translated into that langue. Starting with an initial set of component constraints that correspond to various parts of the body, the library aggregates them into steps, and then motions, exercises and then routines. A library of the various components and the more complex movements and routines can be built up this way in parallel by a team to build a large library of coachable exercises and other movement in a short amount of time.

Building and managing a large library of customizable movement is possible through the use of a componentized movement description language.

Coach Authoring/Editing Tool

A programmer can edit a textual description in the form of a computer language or in the form of data objects, that can be loaded for a particular exercise, parsed, and ‘run’ by the automated coach. For other users, like physical therapists, fitness coaches, and movement guides, an alternative solution to creating automated, coachable movement can be provided through the use of a visual coach/movement authoring tool that offers less technical means of describing motion as follows.

Such a tool enables not only medical practitioners without a programming background to author coachable movement, but also to do it at scale. This addresses the need to create thousands and thousands of variations of exercises that are needed to help people of all ages and in varieties of medical conditions.

Visual Authoring Tool

A Visual Authoring Tool allows visual translation of the language into a screen interface that facilitates non-programmer medical and movement specialists the ability to craft coachable exercises to address specific medical conditions and specific patients' situations.

The visual tool enables describing how the coach application should react given a particular situation. The exercise language includes various constraints, steps, exercises, and routines so that non-programmers can author coachable exercises. This is the Coach Authoring tool.

The tool can provide an animation-like metaphor that may be more familiar to users, and which is easily mapped to the language's movement hierarchy. The tool can describe motion in terms of particular ‘keyframes’ which are called ‘steps’. Another similar metaphor is dance instructions: ‘put your right foot in, put your right foot out, put your right foot in and shake it all about’. The exercise description language and the authoring tools make it possible not only to describe these but to provide and detail the means of describing that movement precisely and quantifiably.

A variety of visual affordances can support unpacking movement into a hierarchical movement representation such as routines, exercises, steps and constraints, or into a non-hierarchical, moment-by-moment representation and other variations that represent movement in sections or sequences. The tool helps by providing affordances that enable a user to specify each representation. The tool affords organizing each representation and to organize them as one can do if they are represented as a part of a movement representation language. In one instance, the tool affords being able create exercises using recorded video and identifying the keyframes. Then the tool provides means to specify the desired body position as an armature, that is; as a rag doll that is captured at the right moment and position. With the description captured as such, the user can also perform verification of the description by running additional videos against the generated movement description and comparing the reported results with those pre-recorded, and also by using the ‘live’ video and performing motion so as to confirm and correct the movement description (e.g., the author can test the authored description through live or recorded performances).

The system may make use of a sequential (or timeline) display of the motion in terms of steps chained together into an exercise, and then, in turn, into a routine. It includes a representation of steps and their constraints that are either directly buildable by the author through the tool or are available as a selectable library of pre-built constraints. That is the tool enables selection of pre-authored constraints without the need to understand the mathematics behind them (e.g., select “arms up”) as part of a step. The system also enables using a pre-recorded video of exemplary movement and scrubbing through it to identify keyframes or steps that matter, and selecting the constraints that define the key aspects of the movement at that time step. One can also switch to live video to confirm that a movement is properly recognized or not recognized, e.g., testing the steps against live video or recorded video.

During process of authoring, constraints' matching statuses are visually highlighted as a means of aiding the author in understanding or debugging how the movement description language is behaving against motion in the aim of specifying conformant motion. The visual indications of constraint matching help tell what aspect of the movement is being recognized and what isn't. The constraints are highlighted in their written out form and/or visually by highlighting the recognized armature/skeleton as displayed or alternatively in representation of the rule visually on the armature, as a targeted position that is currently unmatched and that is overlaid on the live or pre-recorded video stream.

Coach Application

The coach is a patient facing application through which the patient can do routines of exercises at home, like jumping jacks, planks, stretch exercises etc. Using computer vision, the progress and correctness of the exercise is detected. The coach may also provide cues (such as, audio and/or visual indications) to the patient in order to guide them through each step of the exercise so they can execute it correctly.

An editor makes it possible to create new routines using visual elements and feedback, this way there is no need to have programing skills to create the exercises.

As noted above, routines are made of a set of smaller building blocks. The building blocks include constraints, positions, steps, and exercises. Movement can also be sequential, however, this hierarchical approach enables more reusability of the movement description which can be reorganized and reused to construct different movements or simply movement variations.

Constraints are the smallest building block available, they allow the system to measure individual body parts, like an arm or leg. This is done by measuring the distance and angles between keypoints (such as between a joint and the floor, or between joints). For example, if the angle between the left elbow, left wrist, and left shoulder is around 180 degrees, that means the left arm is stretched out.

A position is a collection of constraints translated into a form that masks the mathematics and geometry they contain and yet can be understood by movement and medical practitioners. Some examples of positions could be: both arms stretched up, both legs spread apart, head tilted to the left, bend over, etc. This item is a named collection of constraints that translate into the mathematics and geometry they describe by name.

Thus, a grouping of constraints can be treated as a collective whole. As such they can also be named and reused and aggregated into steps. This allows a recursive decomposition of movement into a means to create descriptions efficiently for the movement conformance engine to operate on.

A step is a set of positions, for example, in a jumping jack, for the starting position, the positions include having both legs together, and having the arms held down pressed against the sides.

An exercise is an ordered set of steps, for example, in jumping jacks, the starting position where the legs are together and the arms are down at the side and an ending positions where the legs are spread apart and the arms are held up.

A routine consists of one or more exercises. For example, a cardio routine may include jumping jacks for a minute, and then run in place for another 5 minutes.

Accordingly, it is possible to match language that healthcare professionals either use directly or can understand easily; however, other terms and decompositions for effective movement description, and could be used by a movement conformance engine to detect and guide such motion. A machine learning model could be trained to generate such a language given a set of movements to be used as training examples.

In general, the model can be used to create a description in the form of a model rather than specific rules. This allows the model to be transformed in order to generate a language that can be reviewed or run to check that designed/detected movement is classified as conformant or not as expected. The classification can include other classes aside from conformant that include warnings, coachable advice that be offered to the use in real time (quickly) or after the movement is performed (when there is more time to coach). The coaching texts can also be generated automatically to correspond to the movement and to the coachable moment in which they can be offered.

Creating an Exercise

The first step in creating a new exercise, is uploading a video in which a person performs the actual exercise (like Jumping Jacks). Key frames from this video will be used to determine the positions the patient should be in while doing the exercise.

FIG. 15 illustrates a step of uploading an exercise video. The user may provide a name of the exercise 1410 and a short description 1420. Additionally, the user may indicate which body parts are targeted 1430 and a type for the exercise 1440. Further tags may also be added 1450.

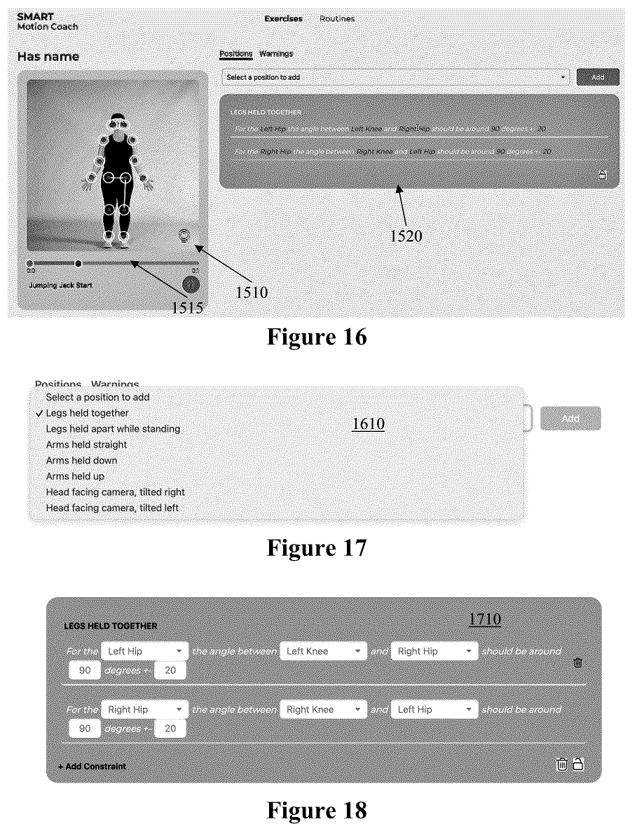

FIG. 16 illustrates a step of defining an exercise step. After the details are filled in, the exercise step definition page appears. On the left of this page, a frame can be selected 1510 from the video by clicking on the timeline 1515. This can be a target for the patient to emulate. (For example, arms down, and feet together at the start of a jumping jack). On the right, various positions are shown in a window 1520.

Then from the available list of positions, a preset can be picked and added as a position target (in this case, legs held together). When the current video frame posture matches the position, the position block will light up green.

When all the positions match (for example, and the user has their arms down and their legs together), the step is matched, and the next step, if any, is checked. (For example, it can be a jumping jack start position and jumping jack end position). These positions can be changed by changing the associated constraints.

FIG. 17 illustrates selecting a position from a list of sample positions. A select window 1610 may be presented which provides a number of position options. The options shown may be filtered based on the system's analysis of the current frame.

Constraints are the fundamental analytic building blocks to determine if limbs or other body parts are in a certain position. This may be done by comparing the angles between different joints, or relative to the world (floor).

When the pose recognition model determines the armature of the user, a multiplicity of angles are available to determine motion conformance, but only a subset are pertinent to the patients care providers and history, and are meant to be saved and available in the form of exercise results or movement assessments. Individual constraints can be marked as important in the exercise position editor for the sake of saving and reporting them.

In addition, the conformance engine records not only moment by moment results but also computes results over time. For example, while the user is correctly holding a given posture (and thus the constraint is matched) the coach will continuously collect the min and max angle for that constraint during the hold time. Or for a dynamic pose, like raising the arm overhead, the engine will record the moving average of an angle selected for reporting. And for an exercise rep, the minimum and maximum squat range can be recorded so that it can be compared from rep to rep.

FIG. 18 illustrates entering definitions for a position. As shown in FIG. 18, the constraint for legs held together 1710 is defined as when the patient is facing the camera, and has the legs together, a 90-degree angle forms on both the left and right side, between the hip, knee and opposing hip. Referring to FIG. 16, these lines are shown drawn between the joints.

This naming is available for reuse and is parameterizable for a given person. For example, an earlier assessment may be performed to determine just how close a person may have their legs together (as well as apart) as some people will have larger thighs or calves. These values can be available also to tune an exercise to their body so that constraint value will use a named minimum or maximum range, say for the legs together, or apart, so that the engine can use these to establish that they are indeed conforming to a position as best they can.

Warnings (a Type of Position)

The conformance engine can check if the patient is doing the exercise correctly, but also can coach or nudge the patient to improve their posture. A set of constraints which we refer to as warnings or interaction triggers. A warning works in a way similar to detecting a target posture but instead of completing the step when matched, these provide a movement author with the option to perform an interface action once they are triggered, such as telling the patient to change their posture or move faster or slower (etc.).

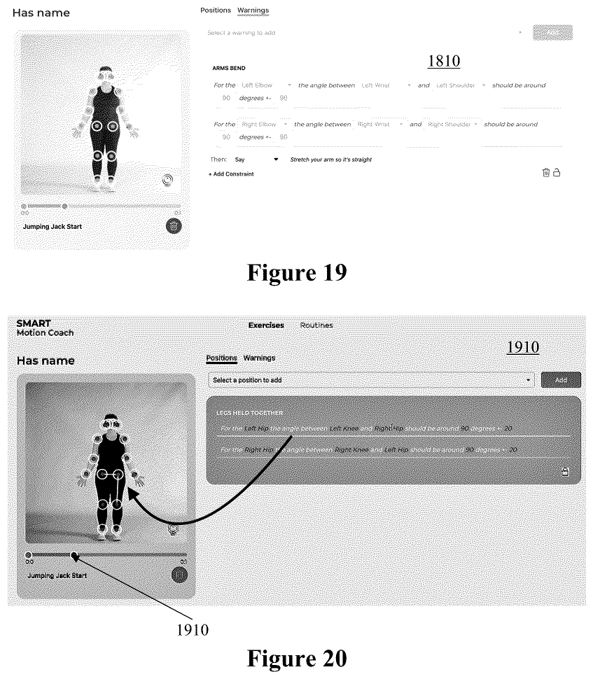

FIG. 19 illustrates entering a warning for a position. As shown in the warning definition 1810 of FIG. 19, the coach can say that they should stretch their arms straight if their arms are bent during the exercise.

Testing the Exercise Step

Once all constraints for a step have been defined, the system can be used to test the results by pressing the camera button. A live video feed from the camera appears in which the same computer vision detection algorithm is applied. So, when the person is in position, the box will light up green. If that doesn't happen, they know that something needs to be changed in the constraints, which can be done live, so feedback is instant.

FIG. 20 illustrates an example of Routine creation. Routines are created by a set of one or more components (such as, speech bubbles and exercises). They can be dragged in the right order using the timeline feature 1910. (Similar to a video editor).

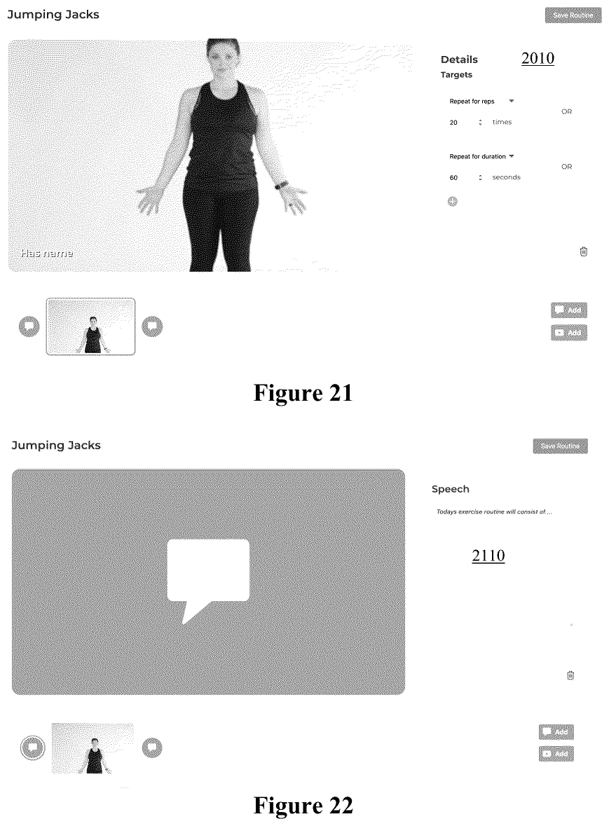

FIG. 21 illustrates entering details for an exercise. Clicking on one of the components (an exercise in the example below) shows the set details 2010 of that component. Here the start of a jumping jacks exercise is selected. The details 2010 indicate how many jumping jacks the patient to do in this routine. Or, if they don't reach it within the allotted time, set a time limit on the exercise. The coach can automatically count each rep out loud.

FIG. 22 illustrates entering speech/text which may be used during an exercise. The speech component allows the coach to say helpful information to the user before or after an exercise. For example, to encourage them on, or to tell them about the motions they are about to be asked to do. Using speech entry 2110, the text of the message can be entered.

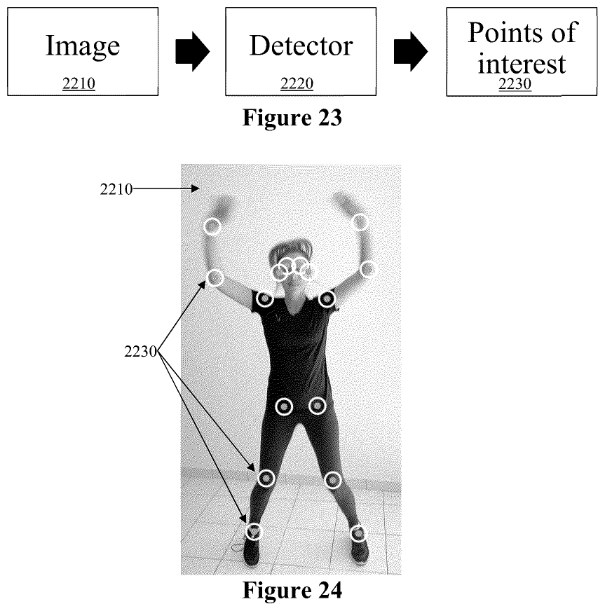

FIG. 23 shows a process flow for an armature recognition. The armature can be detected in images 2210 containing people, or a stream of images (which can, for example, be a video, or a webcam feed). For each image 2210, using a trained model, a list of points of interest are detected by the detector 2220 which identifies various points of interest 2230. The points of interest 2230 can be the relative position of places on a person's body, like a left elbow, right eye, or left foot, as well as real world reference points like a floor, or the relative location of objects which are of interest of an exercise like a ball or chair. The detector 2220 can use a machine learning model to do this. Overall, the input is an image 2210 and the output is a list of items with the name and coordinates of the points of interest 2230.

Additionally other values may be added, such as the confidence in the prediction in the points location, its name, and/or in the visibility of the point. In one example, if the person is viewed from the front, the shoulder blades may be indicated as ‘inferred’ and/or with visibility and confidence values.

FIG. 24 demonstrates a sample armature recognition with points of interest 2230 highlighted. These points of interest 2230 are simple data points with the relative position of where they are in relation to each other. (Seen on a coordinate grid (x,y,(z) 0,0,(0) to 1,1,(1))). Visually these coordinates can be drawn back over the image 2210, to show what is being detected.

The points of interest may be in 2D or 3D space and time. Spatial coordinates can be pixel values (relative to image itself), in absolute spatial values (e.g., meters relative to a physical reference point), or in a relative space with a reference plan with respect to the body center (e.g., ranging from −1 to 1 to distinguish forward from backward in that space).

To each position a label is added, for example left knee, or right ear, floor, etc. This help to easily find the relative location of a specific point of interest. In the case of a sequence of images, these points may be generated for every image in the list, which then is converted into a sequence of lists of points of interest.

As defined above, a constraint is a simple set of rules, which defines the relation between different points of interest on the screen. These can be written in a simple form in which a few key items are defined (like which points of interest to check, and what to check for those specific points). A constraint can be analytic (such as an explicit set of rules) or derived from examples that identify the position (e.g., a machine learning model that classifies and regresses a pose or a subset of points of interest as a position).

Some examples include (but not limited to):
For left elbow, the angle between left shoulder and left wrist should be around 180 degrees. Plus or minus 10 degrees. (Which would be stretching the left arm)
The distance between left foot and right foot should be less than n (to determine the feet are close together)
The distance between floor and ball should be more than a given distance, n (to determine if a ball was kicked high enough)
The left arm and torso points of interest collectively match poses for those patients who have a particular capsular damage within the left shoulder.

These constraints can be written in a way so that they are easy for a computer to parse, but also still for a human to read.

Once the point of interest list is obtained from an image, the system can apply a constraint to that list, which will return whether the constraint is met or not. This can be a yes or no, or a confidence range of how likely it is that the constraint is met.

Some example steps include: 1) position arms held down by side & position feet together for starting pose of jumping jacks; and 2) position arms held up and feet apart for a target pose in jumping jacks.

To determine the likelihood that a step is being matched, each position in the step is evaluated, and a combined result is determined.

For all of the matching results it is also possible to collect an aggregate over time of the matching results. For example, for each video frame in a video over a period of time. This way the system can determine if a step is valid for a longer period of time, such as in the case of holding a plank position over time.

The application can also connect poses together in a sequence or a loop, to create exercises with different postures in a row. At the start, the system can check if the current patients posture matches the starting position, if it matches, it goes to the next step. This process is repeated for each step in the exercise until the exercise is completed.

Once the exercise is completed, the coach application can present the patient with a results page. This page may show details of the exercise, such as completed reps and/or duration of the exercise. Additionally, the page may provide further comments, for example, indicating change from prior experiences, encouragement for further work, suggestions, etc.

Before the loop is completed, different actions may also be performed, such as, saying the current counter count, and saving the counter information. This way the system can save the patients rep count for sets and notify him or her of the progress of the current set.

Once the allotted time or rep count has been achieved, the system can exit the loop and go to the next part of the routine. This sequence can have any number of steps.

Individual patients may benefit from different exercises or variations on an exercise. The language and interface facilitate creating exercises with a degree ease without requiring coding. But also, the language structure of routines/exercises/steps/constraints allows for parameters at each level of performance detail. A routine can include n-number of exercises, each exercise can be repeated n-times, a series of steps constituting a rep-counted loop can be done n-repetitions, the degree of movement described by the constraints can be increased or lessened. Together the parameters can be collected and calibrated to the patient's state, age, gender, diagnostic status, days since injury, days since surgery, etc. This calibration can be done at the will of the medical practitioner, or by the choice of patient, or by an automated methodology that takes into account the patient's medical treatment. Any variety of assessment can be mapped to a set of parameters and movements that optimize for a medical or health goal.

Model-Based Movement Conformance Engine & Authoring

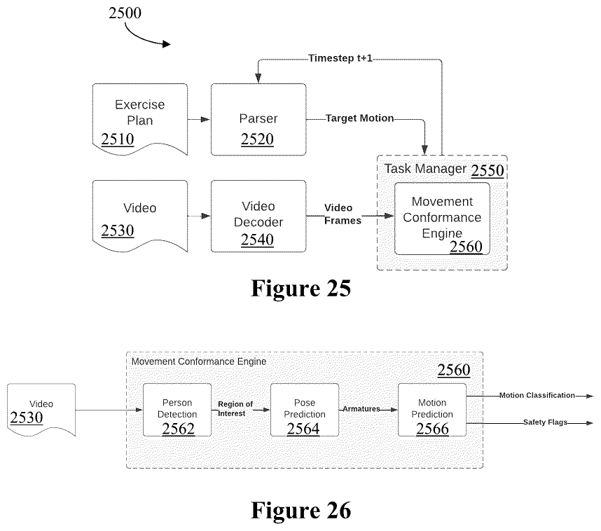

FIG. 25 describes a system architecture at a high level. The Movement Conformance Engine 2560 is harnessed by a set of components that provide it with relevant video frame and target motion data. The “target motion” is the current motion or stance that the user is required to perform before the exercise plan continues.

As shown, the system 2500 includes an exercise plan 2510 (e.g., one stored on a computer memory). The exercise plan 2510 is passed to a parser 2520 that provides target motions to the task manage 2550. A video device 2530 and video decoder 2540 send video frames to the task manager 2550. The video frames and target motion are then analyzed by the Movement Conformance Engine 2560. Once a movement/position is determined, the task manager can instruct the parser 2520 to provide the next target motion for use.

In FIG. 26, the Movement Conformance Engine 2560 of FIG. 25 has been expanded. The motion prediction phase has been described above with an analytical armature matching process. In an alternate embodiment to the analytical approach a model based approach is used.

Similar to the analytical approach, the model-based approach utilizes person detection 2562 and pose estimation models to prepare a sequence of armatures for processing. The person detection 2562 determines a region of interest in the video frames. The pose prediction 2564 can use the region of interest armatures (e.g., by focusing on features within the region of interest) in order to identify which are passed to motion prediction 2566.

One difference is the use of a neural network in place of the hand-crafted algorithm for classifying motions through time. This model takes as input a sequence of N pose armatures and outputs a single motion classification (a particular stance can also be considered a motion) as well as a group of safety flags. These flags can include information about whether the motions can be considered harmful for the user. The safety flags are separate from the motion classification output because certain motions may be considered harmful only under certain scenarios, including the speed at which the motion is acted out.

In the model-based approach, the Motion Prediction algorithm is replaced by a machine learning algorithm which has learned end-to-end how to classify motions and safety flags from example. This uses the construction of a labeled dataset of examples to supervise the model. The dataset is constructed utilizing pairs of armature sequences labeled by a human annotator with the correct motion and safety flags. These motion classification and safety flags are considered the primitives of the movement conformance engine. This is a key difference to the analytical solution, from which arbitrary armatures can be used in the conformance matching process.

The collection of the armature sequence data points can be obtained automatically through video using powerful person detection and pose prediction models, such as the models used in the Movement Conformance Engine 2560, or using other technology such as wearable devices or body scanning technology. Video may be used, as annotation may be easier when looking at a video.

With a dataset constructed, a Movement Prediction model can be trained end-to-end with standard machine learning models, losses and metrics.

Utilizing a model-based approach rather than an analytical approach to classifying motions from a sequence of pose armatures has various advantages:
1. Model-based classifiers are far more accurate and robust to ambiguity;
2. None (or at least fewer) of the steps for classification need to be hand crafted; and
3. Any of the safety flags that require an analytical solution can still exist analytically, parallel to the model-based motion predictor.

In contrast, the model-based approach may include additional work as adding new motion/safety primitives requires more labeled data and adding new motion/safety primitives requires training new models.

Prompt-based Exercise Authoring

A secondary model may be used that is trained with armatures and text so that they can be articulated with words (e.g., using natural language) and the armatures, steps, and constraints are effectively learned by the model and generated to form the overall motion detection (authoring).

The neuro-symbolic approach can be extended with a prompt-based natural language interface. Instead of utilizing a Domain Specific Language for the exercise plan, the user can input a natural language prompt. This prompt is then passed to the Program Synthesizer, which in this case utilizes a large language model (LLM). The LLM is responsible for producing the list of desired motions in this case.

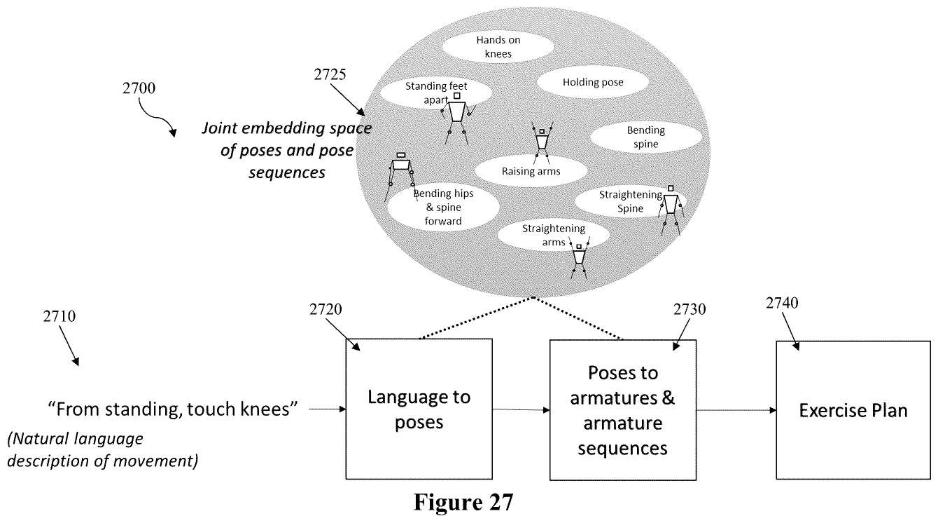

FIG. 27 illustrates using a natural language prompt 2710 to generate an exercise plan (as per 2510) 2740. The natural language process 2700 receives a natural language prompt 2710 (or natural language description). A language to poses module 2720 parses the prompt and determines a sequence of armatures/poses 2730. The armatures/poses may be selected based on a joint embedding space of poses and pose sequences 2725. The determined armatures/poses are then combined to form an Exercise Plan 2740.

Rather than predicting a sequence of motions which are detected individually through time, the system can use a natural language prompt to generate a sequence of armatures that the user must conform to. Postures can be called out at certain moments to articulate examples of non-conformant motion, or motion that can be coached.

The movement conformance engine can either 1) use the heuristic based approach to match armatures through time or 2) utilize motion prediction models on both the armature sequence predicted from the model above (ground truth) and the armature sequence predicted by the pose network

One benefit of this approach is the natural transitions between motions. Some embodiments also implement an armature matching algorithm to ensure they are maneuvering between motions properly. For example, Motion A is followed by Motion B, but there exists Transition A between them. If the user performs Transition A much differently than expected, the system can flag this.

Keypoint labels can be obtained automatically from video using a pose estimation model, through wearable devices utilized by actors acting out our motions primitives, or through body scanning technology. The text classification labels can be declared manually. Multiple text descriptions can be used for each sequence.

One embodiment provides a method for computer-assisted exercise. The method includes loading a definition for the exercise including an ordered list of steps. Each step includes at least one position, and each position is defined by at least one constraint. For each step in the ordered list of steps, the method performs loading a current step, receiving an image of a patient performing the exercise; and determining a plurality of keypoints in the image. For each constraint of the at least one constraint of each position, a determination is made as to whether the constraint is being met by the patient based, at least in part, on the relative locations of the plurality of keypoints. In response to determining a first set of constraints in the position is met by the patient, the method proceeds to a next step in the ordered list of steps.

In a further embodiment of the method above, at least one position associates a second set of constraints with a response. The method also includes in response to determining the second set of constraints is met by the patient, performing a response. The response can include providing audio feedback to the patient; providing visual feedback to the patient; and/or stopping the exercise.

In another embodiment of any one of the methods above, loading the definition for the exercise includes loading a routine defining the exercise and at least one additional exercise.

A further embodiment provides a method for computer-assisted definition of an exercise. The method includes receiving a description of at least one position. The position defines a step and an ordered list of steps defines the exercise. For each position, the method includes determining a plurality of keypoints in an armature for the position, assigning at least one constraint to the position based, at least in part, on the relative locations of the plurality of keypoints and assigning a first set of constraints which, when met, indicate the associated position has been performed. The method also includes storing the at least one constraint as a position in a step; and storing at least one step as part of the exercise.

In another embodiment of the method above, loading an ordered list of images includes loading a video file and selecting at least one image from the video file to be loaded.

In a further embodiment of any one of the methods above, the method also includes receiving a selection of response to be performed and assigning a second set of constraints which, when met, indicate the associated response is to performed.

In another embodiment of any one of the methods above, the description of at least one position includes one of: at least one image showing the at least one position and text describing the at least one position.

In a further embodiment of any one of the methods above, the description of at least one position includes a natural language description of the at least one position.

In another embodiment of any one of the methods above, the description of at least one position includes receiving a video of an expert performing the exercise.

In a further embodiment of any one of the methods above, determining the plurality of keypoints in the armature for the position includes receiving a description of the armature from a library of positions and exercises.

In another embodiment of any one of the methods above, he method also includes storing at least one additional constraint associated with the position. The at least one additional constraint indicates a message is to be given when met. Assigning at least one constraint to the position may include receiving at least one default constraint to the position and adjusting the at least one default constraint for a user to create the at least one constraint to the position.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general-purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method for computer-assisted exercise, the method comprising:
loading a definition for the exercise comprising an ordered list of steps,
wherein each step comprises at least one position, and
each position is defined by at least one constraint, and
for each step in the ordered list of steps:
loading a current step;
receiving an image of a patient performing the exercise;
determining a plurality of keypoints in the image;
for each constraint of the at least one constraint of each position:
determining whether the constraint is being met by the patient based, at least in part, on the relative locations of the plurality of keypoints; and
in response to determining a first set of constraints in the position is met by the patient, proceeding to a next step in the ordered list of steps.

2. The method of claim 1, where at least one position associates a second set of constraints with a response; and
the method further comprises in response to determining the second set of constraints is met by the patient, performing a response.

3. The method of claim 2, wherein the response is one of:
providing audio feedback to the patient;
providing visual feedback to the patient; and
stopping the exercise.

4. The method of claim 1, wherein loading the definition for the exercise comprises loading a routine defining the exercise and at least one additional exercise.

5. A method for computer-assisted definition of an exercise, the method comprising:
receiving a description of at least one position, wherein the position defines a step and an ordered list of steps defines the exercise,
for each position:
determining a plurality of keypoints in an armature for the position;
assigning at least one constraint to the position based, at least in part, on the relative locations of the plurality of keypoints; and
assigning a first set of constraints which, when met, indicate the associated position has been performed;
storing the at least one constraint as a position in a step; and
storing at least one step as part of the exercise.

6. The method of claim 5, wherein loading an ordered list of images comprises loading a video file and selecting at least one image from the video file to be loaded.

7. The method of claim 5, further comprising:
receiving a selection of response to be performed and
assigning a second set of constraints which, when met, indicate the associated response is to performed.

8. The method of claim 5, wherein the description of at least one position comprises one of: at least one image showing the at least one position and text describing the at least one position.

9. The method of claim 5, wherein the description of at least one position comprises a natural language description of the at least one position.

10. The method of claim 5, wherein the description of at least one position comprises receiving a video of an expert performing the exercise.

11. The method of claim 5, wherein determining the plurality of keypoints in the armature for the position comprises receiving a description of the armature from a library of positions and exercises.

12. The method of claim 5, further comprising storing at least one additional constraint associated with the position, wherein the at least one additional constraint indicates a message is to be given when the at least one additional constraint is met.

13. The method of claim 11, wherein assigning at least one constraint to the position comprises: receiving at least one default constraint to the position and adjusting the at least one default constraint for a user to create the at least one constraint to the position.
